(19) United States
(12) Patent Application Publication
(10) Pub. No.: US 2004/0018193 A1
(43) Pub. Date: Jan. 29, 2004

(54) Rapid-acting broad spectrum protection against biological threat agents

(76) Inventors: Ken Alibek, Bristow, VA (US); Charles Bailey, Fayettesville, TN (US); Edith Grene Carron, Leonardtown, MD (US); Serguei G. Popov, Bristow, VA (US); Aiguo G. Wu, Arlington, VA (US); Taissia Popova, Bristow, VA (US); Francis W. Klotz, Manassas, VA (US); Alice Hayford, Silver Srping, MD (US); Vladimir Karginov, Ashburn, VA (US); Qingzhu Zhai, Centerville, VA (US); Ge Liu, Rockville, MD (US)

Correspondence Address: Finnegan, Henderson, Farabow,, Garrett & Dunner, LLP, 1300 I Street, N.W., Washington, DC, 20005, US

(21) Appl. No.: 10/400,062

(22) Filed: Mar. 27, 2003

Related U.S. Application Data
(60) Provisional application No. 60/368,154, filed on Mar. 29, 2002.
(60) Provisional application No. 60/393,555, filed on Jul. 5, 2002.
(60) Provisional application No. 60/393,554, filed on Jul. 5, 2002.
(60) Provisional application No. 60/381,379, filed on May 20, 2002.
(60) Provisional application No. 60/384,116, filed on May 31, 2002.
(60) Provisional application No. 60/428,715, filed on Nov. 25, 2002.
(60) Provisional application No. 60/428,717, filed on Nov. 25, 2002.

(51) Int. Cl. A61K 39/395

(52) U.S. Cl. 424/144.1

(57) ABSTRACT

A treatment for the effects of biological threat agents, such as smallpox virus or anthrax, to be administered either post-infection or as prophylaxis for infection, comprises administration of IFN-α, IFN-γ, or the cell wall of the bacteria B. alcalolophilus, E. faecium, S. caseolyticus, or B. stearothermoohilus or a combination of these components. Additionally, the treatment comprises a combination of antibodies, such as antibodies to heat-inactivated anthrax microbes or to the anthrax Protective Antigen, and antibiotics, such as ciprofloxacin. The treatment also comprises the peptidoglycan, lipoteichoic acid, or muramyl peptide fraction of bacterial cell walls, either alone or in combination with the cytokines, antibodies, and antibiotics provided. These treatments for smallpox and anthrax are administered as an inhalation preparation and therefore avoid the toxic effects of the higher doses of these components. The treatments are also indicated for people at high risk for harmful effects of the smallpox vaccines currently available.

This application claims the benefit of priority of U.S. Provisional Application 60/368,154, filed Mar. 29, 2002 (attorney docket no. 08675-6012); U.S. Provisional Application 60/393,555, filed Jul. 5, 2002 (attorney docket no. 08675-6014); U.S. Provisional Application 60/393,554, filed Jul. 5, 2002 (attorney docket no. 08675-6015); U.S. Provisional Application 60/381,379, filed May 20, 2002 (attorney docket no. 08675-6021), U.S. Provisional Application 60/384,116, filed May 31, 2002 (attorney docket no. 08675-6024), U.S. Provisional Application 60/428,715, filed Nov. 25, 2002 (attorney docket no. 08675-6034), and U.S. Provisional Application 60/428,717, filed Nov. 25, 2002 (attorney docket no. 08675-6035), each of which is incorporated by reference.

This invention was made with Government support under MDA972-01-C-0084 awarded by DARPA and DAMD17-01-C-0033 awarded by the Department of the Army. The Government has certain rights in the invention.

### BACKGROUND OF THE INVENTION

The invention relates to therapies for protection against biological weapons or to therapies for the treatment of the effects of biological threat agents. Biological threat agents are biological agents that can be used as weapons. These include, bacteria, for example, but not limited to, B. anthracis, and viruses, for example, but not limited to, smallpox and human monkeypox. Because it may not be known that there was a biological attack until after the attack has occurred and it may be difficult to determine what agent was used, there is a need in the art for a rapid-acting, broad spectrum therapy.

Anthrax is one of the deadliest human infections. The causative agent of anthrax, Bacillus anthracis, has been used to develop biological weapons stockpiled by the former Soviet Union. The virulence of anthrax is determined mainly by its toxins, which have been the subject of the majority of research on anthrax treatment and prevention. Interest in the pathogenesis and treatment of anthrax has increased in recent years due to concerns over its potential for use as a biological weapon by rogue states or terrorist organizations.

Anthrax pathogenesis is caused by Bacillus anthracis, a gram positive bacteria. The bacteria forms spores that are highly resistant to environmental effects and can remain dormant for decades. Naturally, anthrax spores are found in the harsh environment of the soil. The resistant spores are a good candidate for weapons because they can tolerate milling and processing with additives to make them very small and air-borne and thus more likely to be inhaled and drawn deep into the lungs. After production, their resistance also allows them to be stored in a delivery mechanism, such as a missile warhead, until deployed. Finally, the high mortality rate and lack of an effective defense or treatment make anthrax a good candidate as a weapon. (Dixon, et al., (1999) N. E. J. Med., vol. 341, pp. 815-826.) If effective, easily administered treatments are available, anthrax may become less attractive to terrorists, and the risk of an attack might be reduced.

There are very few known treatments for anthrax infection. Because the disease is so rare, few treatments or therapies have been explored. A vaccine against anthrax for humans or livestock is available, but it is not the final answer to the problem of an effective response against a biological terrorist attack. The vaccine is an aluminum hydroxide-precipitated preparation of protective antigen (PA) from the attenuated, non-encapsulated Sterne strain of B. anthracis. It requires multiple injections over a period of eighteen months. (Dixon, et al., (1999) N. E. J. Med., vol. 341, pp. 815-826.) Therefore, the vaccine is likely to be most effective when administered long before exposure, not after the surprise of a terrorist attack. In addition, there are concerns of serious side effects of the anthrax vaccine currently available.

The ability of the non-immunized population to respond effectively to an attack with aerosolized B. anthracis spores crucially depends on four major factors: (i) efficient prophylaxis of infection before the exposure to the agent, (ii) prevention of the disease in the exposed people, (iii) treatment of the infected persons, (iv) post-treatment prophylaxis to guard against disease resulting from germination of latent spores after cessation of antibiotic treatment.

In general, two main factors play a role in anthrax infection: cytotoxic effect of lethal toxin and the accumulation of anthrax bacilli leading to a significant change in mass exchange characteristics, such as oxygen and nutritional substance deprivation, and accumulation of various bacterial and host toxic products with eventual organ failure and death.

Large-scale immunization against anthrax, similar to that against smallpox, is not acceptable for several practical reasons including potential problems with a protective efficacy and reactogenicity of anthrax vaccine. Currently, there is no effective treatment for inhalational anthrax, the form most likely to be seen in a biological attack, beyond the administration of antibiotics shortly after exposure to anthrax spores (Inglesby et al., 1999). As illustrated by the recent anthrax attacks in the United States, administration of antibiotics during the nonspecific prodromal period can lead to increased survival, but is ineffective after specific symptoms are manifested (Jernigan et al., 2001). However, side effects of antibiotics prohibit their prophylactic and post-exposure use for a large number of people belonging to different medical risk groups. Furthermore, by the time specific symptoms appear it is often too late for antibiotics to be effective. It is also conceivable that antibiotic resistant strains of anthrax could be used in possible future attacks. Therefore, there is a need in the art for supplements to traditional antibiotic intervention with a new safe and efficient treatment. These safe, effective, and simple treatments are especially important in the case of agents of biological warfare to ensure the public that the situation is under control, thus preventing wide-spread panics that might be as, or even more, damaging than the actual pathological effects of an attack. The invention is intended to contribute to this goal.

Smallpox is another of the most dangerous potential biological weapons. Routine vaccinations for smallpox were discontinued in the United States in 1972, and the last documented naturally occurring case of smallpox was recorded in Somalia in 1977. In 1980, the World Health Assembly declared smallpox eradicated. It recommended that all routine smallpox vaccinations be suspended on a global scale and mandated that reference samples be retained only in two locations, the United States and the former USSR. All other smallpox stocks were to be destroyed.

Vaccination is an effective means for pre- and post-exposure prophylaxis against smallpox. For post-exposure prophylaxis, the vaccine must be administered within 4 days of exposure (Henderson et al., 1999). Although effective prophylaxis, post-vaccination complications have been reported in 14 to 52 people per million, and 1 or 2 of these people may die from the vaccine. Those with deficient immune systems appear to be especially at risk. Complications include eczema vaccinatum, generalized vaccinia, ocular vaccinia, and progressive vaccinia. Complications of vaccination may be treated by administering vaccinia immune globin (VIG) obtained from vaccinated people, although this reagent is in short supply and may present safety concerns, such as contamination with other viruses, itself.

In addition to vaccination, the antiviral drug cidofovir may have some use in treating smallpox infections. Cidofovir has been shown, though, to protect mice against lethal cowpox challenge (Martinez et al., 2000). It may not be optimal, though, for patients with immune deficiency and there are difficulties in oral administration of the drug. There exists a need in the art for new, safe, and effective treatments for smallpox infection.

In addition to naturally-occurring pathogens such as anthrax or smallpox, agents used by bioterrorists may be genetically engineered to resist current therapies and evade vaccine-induced immunity. New therapies, such as the application of antiviral and immunomodulator cytokines, represent promising methods of prophylaxis or treatment, which might avoid these problems (Xing and Wang, 2000; Karpov, 2001; Melian and Plosker, 2001). For instance, the interferons (IFNs) have been used to treat patients with hepatitis virus (HBV and HCV) infections (Lai and Wu, 2000; Moradpour and Blum, 1999). There also exists a need in the art for specific treatments for threat agents such as anthrax and smallpox that cannot be evaded by a genetically engineered virus and that do not present harmful side effects.

When an infection occurs, the body has an initial, rapid response to it. Before specific antibodies are produced, a branch of the immune system called the “innate immune system” is activated to produce the first proinflammatory reaction. In addition, the innate system instructs the other branch of the immune system, the “adaptive system,” to begin to produce the specific responses of T-cells and B-cells and the production of antibodies. Control of the innate immune system offers many opportunities for intercepting infections in their early stages and preventing some of the most severe effects, such as septic shock.

One aspect of the innate immune system that provides protection against viruses is the action of the antiviral cytokines. IFN-α/β protect neighboring cells from being infected by the same or unrelated viruses (Biron, 1997; Kontsek and Kontsekova, 1997). Mice lacking receptors for IFN-α/β and/or for IFN-γ demonstrate reduced resistance to poxvirus infections (Van den Broek, 1995; Biron, 1998). In addition to their direct antiviral activities, cytokines display immunomodulatory functions (Biron, 1997; Xing, 2000). For instance, IFN-γ, IL-2, and IL-12 are potent activator of macrophages and NK cells (Karupiah et al., 1991; Ramshaw et al., 1997). Vaccination of monkeys with simultaneous administration of IFN-Ε/β or the IFN inducer poly(l):poly(C) induced immunity that prevented viremia and alleviated local inflammatory reactions caused by vaccination (Bektemorov et al., 1980).

The cytokine granulocyte-macrophage colony-stimulating factor (GM-CSF) has also been extensively studied as a multipotent immunostimulating substance. It displays a number of broad-spectrum beneficial therapeutic properties. A new drug called Leukine® is based on yeast-expressed human recombinant GM-CSF. Studies demonstrated a beneficial effect of GM-CSF administration for a number of bacterial infections. It has low toxicity, can be administered by different routes, and is well tolerated by patients causing a small number of side effects. The properties of GM-CSF that make it a very attractive candidate drug for adjunct therapy and prophylaxis together with antibiotics against biological weapon agents, however it has never before been evaluated in for this purpose.

Neutrophils, monocytes, and tissue-based macrophages are major cellular components of the innate immune system. Four cytokines, granulocyte colony-stimulating factor (G-CSF), GM-CSF, macrophage colony-stimulating factor (M-CSF), and interferon-gamma (IFN-γ), have received increasing attention as potential adjunctive immunomodulatory agents for treatment of infectious diseases. Studies conducted in vitro and in vivo have shown that these cytokines can augment the functional antimicrobial activities of neutrophils. Similarly, GM-CSF, M-CSF, and IFN-γ up-regulate multiple antimicrobial mechanisms in monocytes and macrophages. Studies conducted in animal models have shown the potential use of each of these cytokines for the treatment of infections caused by a variety of bacterial, fungal, and parasitic diseases. However, clinical experience with these immunomodulatory cytokines is relatively limited, and controlled clinical trials are necessary to define specific indications for the administration of these cytokines in therapeutic regimens (for reviews see Liles, 2001; Armitage, 1998).

Stimulation of murine macrophages with TNF-α, IFN-γ, and GM-CSF, but not M-CSF, was associated with mycobacteriostatic and/or mycobactericidal activity in macrophages. Treatment with these cytokines at 24 h prior to infection with mycobacteria was considerably more effective than treatment after the beginning of infection (Hsu et al., 1995). In another study, treatment of murine macrophages with murine GM-CSF for 24 h enhanced their capacity to restrict growth of C. albicans (Yamamoto et al., 1997). A combination of GM-CSF with antibiotics (amikacin or azithromycin) was associated with a significant increase in killing of Mycobacterium both within cultured macrophages and in infected mice (Bermudez et al., 1994). In a small pilot study, human recombinant GM-CSF (sargramostim) appeared to exert a beneficial effect on the mucosal mycoflora and was suggested as a possible adjunctive therapy in the management of fluconazole-refractory mucosal candidiasis in advanced HIV-positive patients (Vazquez et al., 2000). Singh and Singh (2001) reported a significant suppression of the parasitaemia after co-administration of GM-CSF and met-enkephalin against blood-induced Plasmodium berghei infection in Swiss mice, apparently through macrophage-mediated mechanisms. GM-CSF in combination with appropriate antibiotics was found to be an effective and safe treatment for the management of patients with pneumonia and severe hematopoietic dysfunction (Dierfort et al., 1997). In Phase III trial subcutaneous injections of GM-CSF three times per week for 24 weeks significantly reduced the incidence of overall infections and delayed time to first infection in HIV patients (Angel et al., 2000).

NO is a short-lived and short-distance radical gas that can diffuse easily in the absence of specific cellular receptors and provides macrophages with cytolytic or cytotoxic activity against microbes during infection and inflammation (MacMicking, Xie, et al. 1997). NO functionally inhibits enzymes requiring iron and sulfur prosthetic groups by forming nitrosyl-iron-sulfur complexes (Harris, Buller, et al. 1995), and may inhibit ribonucleotide reductase, an enzyme required for viral DNA synthesis (Lepoivre et al. 1991; Kwon et al. 1991). The latter has been proposed as a mechanism to explain NO-mediated inhibition of VV replication (Karupiah & Harris 1995; Melkova & Esteban 1994). NO is produced by inducible NO synthase (iNOS), which catalyzes the conversion of L-arginine to NO and L-citrulline. iNOS gene expression in macrophages is regulated by cytokines and microbial products via transcriptional induction (MacMicking et al. 1997). Elevated iNOS gene expression has been associated with increases in NO production. (Reiss & Komatsu 1998.) Activation of macrophages with IFN-γ and/or LPS results in increased iNOS expression and inhibition of replications of poxviruses, herpesviruses, retroviruses, and flaviviruses. (Akarid et al. 1995; Croen 1993; Harris 1995; Lin et al. 1997.) NO release has been shown to be inhibited in the presence of anti-CD14 antibody in PGN activated mice (Pugin et al. 1994), as neutralization of CD14 with its antibody blocks NO induction. (Pugin, Heumann, et al. 1994.)

It has previously been shown that NO-mediated inhibition of viral replication is neither host cell nor virus specific (Harris et al. 1995), and that IFN-γ synergizes with IFN-α/γ and TNF-α in NO induction. For mouse peritoneal macrophages, IFN-γ is the only agent reported to date that has been shown to effectively induce NO when tested alone (Ding et al. 1988). A recombinant vaccinia virus expressing iNOS has been found to be attenuated in vivo (Rolph et al. 1996). It has been speculated that NO induction in macrophages is one of the important antiviral strategies in infectious loci, where neutralizing antibodies are unavailable and macrophages are able to ingest and destroy the immature virus particles, thus preventing prevent virus dissemination (Harris et al. 1995). However, NO production may contribute to the control of VV growth, but other antiviral mechanisms, in the absence of NO, are able to mediate virus clearance. This hypothesis is based on the observation that treatment of VV-infected mice with an iNOS inhibitor does not affect the course of infection (Rolph et al. 1996; Rolph et al. 1996). It remains unclear whether the NO cytotoxic effector molecule plays an important role in human macrophage antimicrobial or antitumor activities, as it is difficult to show that human monocytes produce NO following cytokine activation.

Innate immunity against bacterial infections is based on “germline-encoded pattern recognition receptors” that sense the invading pathogen. (Takeuchi and Akira 2001.) These receptors are able to sense the specific characteristics of pathogens, such as the components of the bacterial cell wall, to determine what initial response would be the most effective. Sensing of the characteristics of the invading pathogen is achieved through receptors called Toll-like receptors (TLRs), which are highly conserved through evolution. (Takeuchi and Akira 2001.) The TLRs provide the specific parameters for responses to infection.

The responses mediated by the TLRs include the activation of monocytes/macrophages and the production of TNF-α, IL-1, IL-6, and IL-12, nitric oxide (NO), and eichosinoids. These responses are all part of the proinflammatory reaction and are also signals for the beginning of adaptive immunity. Too much production of these proinflammatory responders, though, may cause septic shock and death.

In addition to the contribution of the different TLRs, the components of bacterial cell walls activate the TLRs in specific ways and achieve specific responses from the TLRs. LPS, a component of Gram-negative bacterial cell walls, lipoproteins, peptidoglycan, lipoteichoic acid, unmethylated DNA with CpG motifs, lipoarabinomannan, N-formly-Met, mannans, mannoproteins, zymosan, and heat shock proteins have been shown to be activators of different TLRs. (Aderem and Ulevitch 2000.) Different pathogen-associated molecular patterns, or PAMPs, may be created by different bacterial pathogens and elicit different cohorts of TLRs for recognition and immune responses.

The bacterial genus Bacillus, as well as other genera provide a potential source of PAMPs, which can be mapped and exploited. Individual species of Bacillus provide specific responses in the innate immune system. The components of the cell walls responsible for these responses can be purified and used to elicit these specific responses on demand. Specifically, the species B. alcalophilus is a potential source of PAMPs that can be used to modulate the immune system in the treatment of bacterial infections, including infections by B. anthracis and smallpox.

Bacteria of the genus Bacillus typically have cell wall peptidoglycans of the directly cross-linked meso diaminopimelic acid (meso-DAP or mA2pm) type. The exact peptidoglycan of B. alcalophilus is unclear. The main menaquinone of B. alcalophilus is MK-7. (Claus and Berkeley 1986.)

Currently, there are few effective treatments for infections of biological threat agents. In light of the recent terrorist events, and among concerns over the possible proliferation of smallpox and anthrax weapons technology to rogue states, the search for ways to treat these and other infections of bioterrorism has become especially important in the art.

### SUMMARY OF THE INVENTION

The invention solves the need in the art by providing treatments for the effects of biological weapons such as, but not limited to, anthrax and smallpox, comprising a rapid-acting, broad spectrum therapy. In general, these treatments comprise compositions of cytokines and optionally bacterial cell walls or components of bacterial cell walls.

While the cytokine and cell wall components are effective as therapies when administered individually, they are also effective in combination therapy and can provide synergistic effects in combination. Combination therapy includes the administration of two or more of these active substances in admixture, or sequential administration of the active substances, one after the other.

In embodiments of the invention cytokines include, but are not limited to, IFN-α, IFN-γ, and GM-CSF. The species of bacteria include, but are not limited to B. alcalophilus, E. faecium, S. caseolyticus, or B. stearothermophilus. The invention also provides for treatments with compositions comprising fractions of the cell walls, including but not limited to, the peptidoglycan, lipoteichoic acid or muramyl peptide fractions.

An embodiment of the invention also includes compositions that further comprise synergistically acting antimicrobial cytokine combinations, computer modeling of cytokine-receptor interactions for rational design of peptide/small molecule mimetics, and small molecule innate immunoligands (SMILs) as antimicrobial agents.

In another embodiment of the invention the composition further comprises antibodies to live or inactivated B. anthracis or components of B. anthracis bacteria and antibiotics.

### BRIEF DESCRIPTION OF THE DRAWINGS

This invention will be described in greater detail by reference to the drawings in which:

FIG. 1 depicts antiviral activity of cytokine- or crude cell wall-stimulated murine macrophages (RAW 264.7) against vaccinia virus-infected human 293 cells, as measured by total PFU of virus present in the sample. CW1=Bacillus anthracis cell wall; CW2=Staphylococcus caseolyticus cell wall.

FIG. 2 depicts antiviral activity of cytokine- or crude cell wall-stimulated primary murine splenocytes against vaccinia virus-infected murine Colon26 cells, as measured by total PFU of virus present in the sample. CW1=Bacillus anthracis cell wall; CW2=Staphylococcus caseolyticus cell wall.

FIG. 3 depicts nitric oxide production by cytokine-stimulated murine macrophages (RAW 264.7), as determined using the Greiss reaction.

FIG. 4 depicts target cell cytolysis by primary murine splenocytes from mice treated for 5 days with cytokine or crude bacterial cell wall preparation, as determined using a standard ⁵¹Cr release assay with Colon-26 cells as the target. CW1=Bacillus anthracis cell wall; CW2=Staphylococcus caseolyticus cell wall.

FIG. 5 depicts CD25 marker expression by primary murine splenocytes from mice treated for 5 days with cytokine or crude bacterial cell wall preparation, as determined using antibody staining and flow cytometry. CW1=Bacillus anthracis cell wall; CW2=Staphylococcus caseolyticus cell wall.

FIG. 6 depicts nitric oxide production by murine peritoneal exudate cells from mice treated for 5 days with cytokine or crude bacterial cell wall preparation, measured as nitrite using the Greiss reaction. CW1=Bacillus anthracis cell wall (25, 50, or 100 μg/ml); CW2=Staphylococcus caseolyticus cell wall (25, 50, or 100 μg/ml).

FIG. 7 depicts survival of VEE virus-infected mice treated with various cytokines. Mice were treated with cytokines on days −2, 0, +2, and +4 and infected on day 0 with 25 PFU of VEE virus.

FIG. 8 depicts nitric oxide production by murine macrophages (RAW 264.7) exposed to IL-15 or IFN-γ alone or in combination with the nitric oxide synthase inhibitor L-NMA (100 or 300 μM), as determined using the Greiss reaction.

FIG. 9 depicts antiviral activity of murine macrophages (RAW 264.7) exposed to IL-15 or IFN-γ alone or in combination with the nitric oxide synthase inhibitor L-NMA (100 or 300 μM) against vaccinia virus-infected human 293 cells, as measured by titration the virus titer of the sample (Mean±SD).

FIG. 10 depicts superoxide anion production by human neutrophils isolated from PBMC (Donor 1) and treated-with cytokines. Superoxide anion levels were measured by SOD-inhibitable reduction of ferric cytochrome c.

FIG. 11 depicts superoxide anion production by human neutrophils isolated from PBMC (Donor 2) and treated with cytokines. Superoxide anion levels were measured by SOD-inhibitable reduction of ferric cytochrome c.

FIG. 12 depicts the production of IL-8 by cytokine-stimulated human neutrophils isolated from peripheral blood, as measured by ELISA.

FIG. 13 depicts the production of IL-8 by human neutrophils isolated from peripheral blood and stimulated with GM-CSF, as measured by ELISA.

FIG. 14 depicts survival of cytokine-treated A/J mice infected intraperitoneally with 5×10⁵ spores of B. anthracis (Sterne). Cytokines (2×10⁴ U GM-CSF, 100 ng IL-12, or 10⁴ U IFN-γ) were administered intranasally on days −2, 0, +2, and +4 of infection.

FIG. 15 depicts dynamic trajectory shapes for TNF-A, -B, and -R.

FIG. 16 depicts a dynamic complex model of TNF-A and TNF-R.

FIG. 17 depicts production of TNF-α (pg/ml) by murine (DBA/2J) peritoneal exudate cells activated by BACW—B. anthracis cell wall, 1 μg/ml; LPS-0.1 μg/ml; PG—peptidoglycan from B. anthracis cell wall, 10 μg/ml; TA—teichoic acids from B. anthracis cell wall, 10 μg/ml. Murine cells were activated with IFN-γ (10 U/ml) 24 h prior stimulation and cytokine release was detected 48 h after stimulation.

FIG. 18 depicts production of IL-6 (pg/ml) by murine (DBA/2J) peritoneal exudate cells activated by BACW—B. anthracis cell wall,1 μg/ml; LPS-0.1 μg/ml; PG—peptidoglycan from B. anthracis cell wall, 10 μg/ml; TA—teichoic acids from B. anthracis cell wall, 10 μg/ml. Murine cells were activated with IFN-γ (10 U/ml) 24 h prior stimulation and cytokine release was detected 48 h after stimulation.

FIG. 19 depicts production of NO measured as nitrite (μM of NO₂⁻) by murine (BALB/c) peritoneal exudate cells activated by BACW—B. anthracis cell wall, 1 μg/ml; LPS-0.1 μg/ml; PG—peptidoglycan from B. anthracis cell wall, 10 μg/ml; TA—teichoic acids from B. anthracis cell wall, 10 μg/ml. Murine cells were activated with IFN-γ (10 U/ml) 24 h prior stimulation and cytokine release was detected 24 h after stimulation.

FIG. 20 depicts IFN-γ levels in plasma of mice administered crude cell wall preparations or LPS intraperitoneally daily for 5 days. Cell wall #1=Bacillus anthracis cell wall; Cell wall #2=Staphylococcus caseolyticus cell wall.

FIG. 21 depicts IL-6 levels in plasma of mice administered crude cell wall preparations or LPS intraperitoneally daily for 5 days. Cell wall #1=Bacillus anthracis cell wall; Cell wall #2=Staphylococcus caseolyticus cell wall.

FIG. 22 depicts virus titer (left axis) and nitric oxide production (right axis) in murine macrophages (RAW 264.7) stimulated with cell walls. The nitric oxide scale is inverted to illustrate the direct correlation between increased nitric oxide production and decreased virus titer.

FIG. 23 depicts superoxide anion production by human neutrophils isolated from PBMC and treated with cell wall. Superoxide anion levels were measured by SOD-inhibitable reduction of ferric cytochrome c.

FIG. 24 depicts the production of IL-8 by human neutrophils isolated from peripheral blood and stimulated with cell walls, as measured by ELISA.

FIG. 25 depicts IL-8 production (left axis) and lactoferrin production (right axis) by human neutrophils isolated from peripheral blood and stimulated with cell walls, as measured by ELISA.

FIG. 26 depicts protection of RAW 264.7 cells from LeTx-induced cell death by anti-PA monoclonal antibody. LeTx, in the form of 64 ng/ml LF+500 ng/ml PA, was added to all groups except the group labeled Control. Controls were RAW 264.7 cells alone with no LeTx and no PA mAb (“Control”) and RAW 264.7 cells with LeTx and no PA mAb (“0 mkg/ml”). Cell viability was determined by MTS calorimetric assay. Error bars represent confidence intervals.

FIG. 27 depicts protection of RAW 264.7 cells from LeTx-induced cell death by anti-PA polyclonal antibodies. LeTx, in the form of 64 ng/ml LF+500 ng/ml PA, was added to all groups except the “Control” group. Controls were RAW 264.7 cells alone with no LeTx and no PA pAb (“Control”) and RAW 264.7 cells with LeTx and no PA mAb (“0 mkg/ml”). Cell viability was determined by MTS calorimetric assay. Error bars represent confidence intervals.

FIG. 28 depicts protection of DBA mice against anthrax infection using the antibiotic ciprofloxacin in combination with rabbit antibodies against protective antigen and inactivated bacteria. Twelve week old female DBA/2 mice (obtained from Charles River, Wilmington, Mass.) were inoculated with 1×10⁷ spores of the Bacillus anthracis Sterne strain by intraperitoneal administration. Five hours after infection, the mice were injected i.p. with 10 mg/kg of anti-protective antigen (PA) IgG, anti-heat inactivated bacteria (HIB) IgG, or a combination of both antibodies. On days 2 and 3 the mice were given two injections per day (morning and late afternoon) of the antibodies. On days 4-7, mice were injected with antibodies once a day. The antibiotic ciprofloxacin was also administered (50 mg/kg) subcutaneously to each mouse once a day for 10 days. The mice were monitored daily. The treatments are as follows: ▴—Stern strain only; ●—ciprofloxacin; Δ—anti-PA IgG; x—anti-PA IgG and ciprofloxacin; ∘—anti-PA IgG and anti-HIB IgG; ▪—antiHIB IgG, anti-PA IgG, and ciproflixacin; □—anti-HIB IgG and ciprofloxacin.

FIG. 29 depicts survival rates of mice pretreated with IFN-α or IFN-γ and infected with vaccinia virus.

FIG. 30 depicts survival rates of mice pretreated with IFN-γ or GM-CSF and infected with anthrax.

FIG. 31 depicts survival rates of mice treated with bacterial cell walls from various types of bacteria and infected with B. anthracis (Sterne strain). “CW33” represents B. alcalophilus, while “CW8” and “CW20” represent E. faecium and A. crystallopoietes, respectively.

FIG. 32 depicts CFU/spleen in A/J mice challenged with B. anthracis (Sterne strain) and treated with B. alcalophilus cell wall. CW33 indicates B. alcalophilus.

FIG. 33 depicts the ability of IFN-γ or the cell wall of B. alcalophilus, either alone or together, to inhibit vaccinia virus replication. “Cytokine 1” is IFN-γ and “PG33” is B. alcalophilus cell wall.

FIG. 34 depicts the effects of bacterial cell walls or IFN-γ on VV replication. RAW 264.7 cells were activated with IFN-γ (100 U/ml) or cell wall preparation from indicated bacteria (1 μg/ml) for 20 hours. The effector cells were co-cultured with human 293 target cells infected with VV (MOI=1) for 20 hours. The mixtures were subjected to three cycles of freezing/thawing and viruses were titrated by plaques assay. The results were expressed as mean of three separate experiments, each containing triplicate samples. Error bars represent standard error of means.

FIG. 35 depicts the effects of IFN-γ plus bacterial cell walls on VV replication. RAW 264.7 cells were activated with IFN-γ (100 U/ml) plus each cell wall preparation from indicated bacteria (1 μ/ml) for 20 hours. The effector cells were co-cultured with human 293 target cells infected with VV (MOI=1) for 20 hours. The mixtures were subjected to three cycles of freezing/thawing and viruses were titrated by plaques assay. The results were expressed as mean of three separate experiments, each containing triplicate samples. Error bars represent standard error of means.

FIG. 36 depicts the effects of peptidoglycan (PGN) or cell wall of Baccillus alcalophilus on VV replication. RAW 264.7 cells were treated with cell wall (CW, 1 mg/ml) or various amount of PGN (μg/ml) from Baccillus alcalophilus (1 μg/ml) for 20 hours. The effector cells were co-cultured with human 293 target cells infected with VV (MOI=1) for 20 hours. The mixtures were subjected to three cycles of freezing/thawing and viruses were titrated by plaques assay. The results were expressed as mean of triplicate samples. Error bars represent standard deviation.

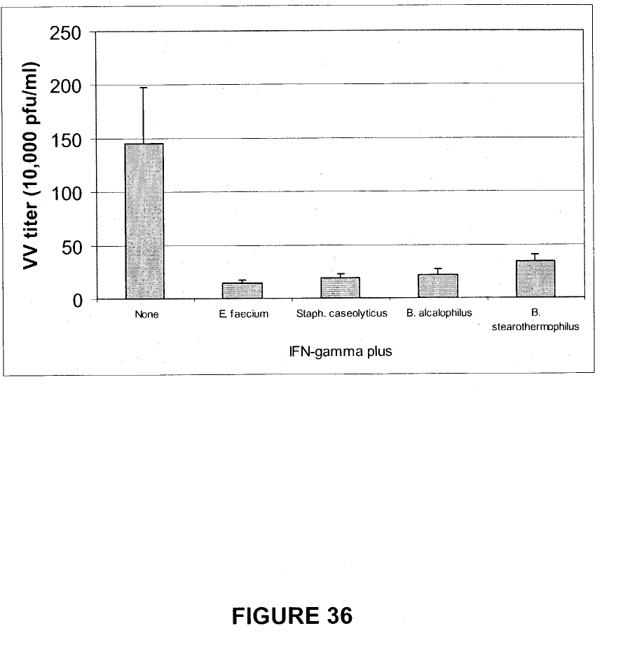

FIG. 37 depicts the effects of peptidoglycan (PGN) or cell wall of Baccillus alcalophilus on nitric oxide (NO) release. RAW 264.7 cells were treated with cell wall (CW, 1 mg/ml) or various amount of PGN (μg/ml) from Baccillus alcalophilus (1 μg/ml) for 20 hours. The culture media (100 μl) were mixed with equal volume of Griess reagent. Following 3-hour incubation, OD₅₅₀ was read and NO concentration (μM) was calculated from a standard curve of nitrate acetate. The results were expressed as mean of triplicate samples. Error bars represent standard deviations.

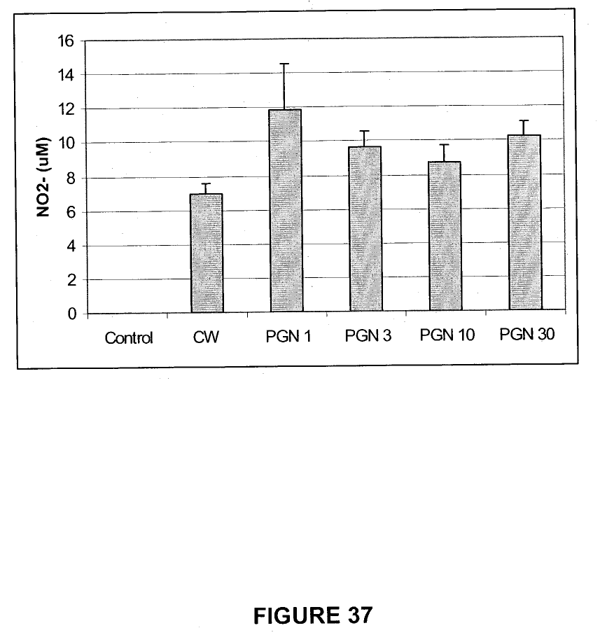

FIG. 38 depicts protection of DBA/2 mice against anthrax infection using ciprofloxacin in combination with sheep antibodies against protective antigen and live bacteria. Twelve-week-old female DBA/2 mice were inoculated with 1×10⁷ spores of the Bacillus anthracis Sterne strain by intraperitoneal route. Five hours after infection, the mice were injected i.p. with 10 mg/kg of anti-PA IgG, anti-BA IgG. On days 2 and 3 the mice were given two injections per day (morning and late afternoon) of the antibodies. On days 4 to 10, mice were injected with antibodies once a day. Ciprofloxacin was administered (50 mg/kg) subcutaneously once a day on days 2 to 10. The delayed treatment (D) groups were treated with anti-PA antibodies once a day on days 2 to 10. The mice were monitored daily. The symbols indicate the following treatments: ▴—Sterne strain B. anthracis; ⋄—control (irrelevant) IgG; Δ—anti-PA IgG; ♦—anti-BA IgG; ●—ciprofloxacin; X—anti-PA IgG plus ciprofloxacin; □—anti-BA IgG plus ciprofloxacin; [symbol] —vehicle control; [symbol] —anti-PA IgG plus ciprofloxacin, delayed; [symbol] —anti-PA IgG, delayed.

### DETAILED DESCRIPTION OF THE PREFERRED EMBODIMENTS

Studies of Synergistically-Acting Antimicrobial Cytokine Combinations

Assays of macrophage-mediated vaccinia virus clearance, in which cytokine-activated mouse macrophage cells (Raw264.7) are assayed for their ability to inhibit replication of vaccinia virus in human KB cells according to a published procedure were performed. An approximately 50% reduction in virus titers was observed in macrophages activated by IFN-γ plus IFN-β and by IFN-γ plus TNF-α. These results show that there is a synergetic antiviral effect between IFN-γ and IFN-β, and between IFN-γ and TNF-α. In addition, NO₂⁻ production from macrophages activated by cytokines was monitored and a synergetic effect in NO₂⁻ production was observed from macrophages activated by IFN-γ and IFN-β. NO₂⁻ was demonstrated to mediate antiviral activities for several viruses, including poxviruses and flaviviruses.

Splenocytes from pathogen-free mice (BALB/c or CBA) were activated by cytokine for 24-48 hours and mixed with vaccinia virus-infected mouse L-929 cells. The results of the first experiment showed approximately 50% reduction in virus titers from macrophages activated by IFN-β at 24-h activation, and from those activated by TNF-α at 48-h activation. Dose titration demonstrated that the low dose (30 units per ml) cytokine worked best compared to higher doses (100-300 units/ml).

In vivo studies of cytokine were performed. These studies involved administering IL-2, IL-12, IL-15, IL-18, GM-CSF, TNF-α, IFN-γ, and IFN-α/β to mice and harvesting bronchial-alveolar lavage (BAL), whole blood, and spleens. BAL, spleen cells, and mononuclear cells from whole blood are used to assay activation and/or function by at least two of the following methods:

| FUNCTION: | ACTIVATION: |
|---|---|
| Phagocytosis | CD69 expression |
| Bacterial killing | CD25 expression |
| Nitric Oxide (NO) production | |
| Viral clearance | |
| NK/LAK assay | |

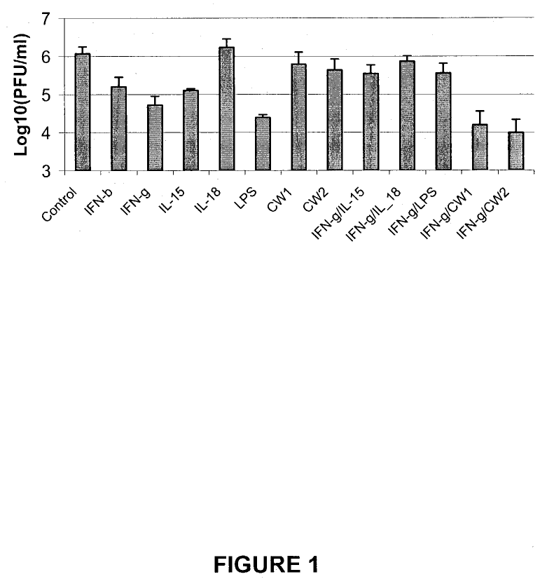

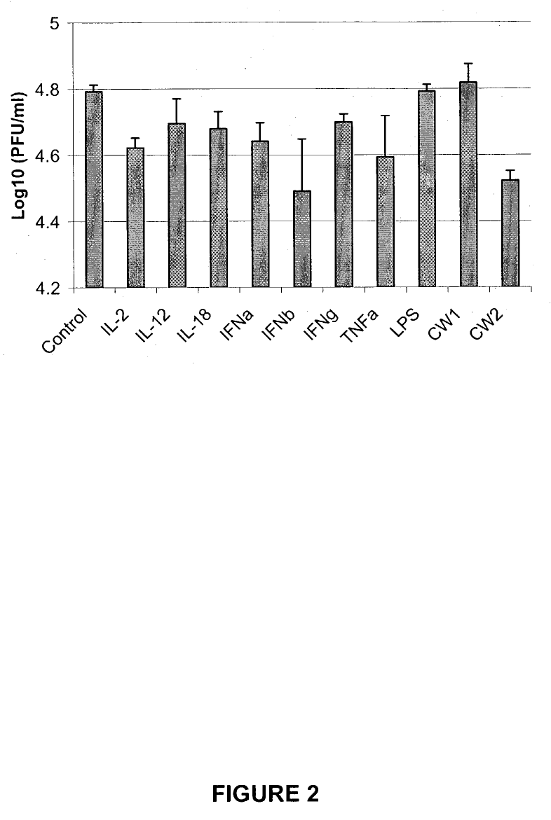

In vitro studies of vaccinia virus clearance mediated by cytokine-activated murine cells were performed. Assays of vaccinia virus clearance using the murine macrophage cell line RAW 264.7 as effectors and virus-infected 293 cells (human cell line) as targets indicated that IFN-β, IFN-γ, and IL-15 reduced total virus titer significantly (by more than 50%) (FIG. 1). Primary murine splenocytes as effectors and virus-infected Colon-26 cells (murine cell line) as targets showed significant effects only for IFN-β (FIG. 2). These different results most likely reflect the differences inherent in the target cells, namely in the interactions between MHC-I molecules and NK receptors. In addition, the use of human cell targets avoids species-specific cytokine effects on the target cells.

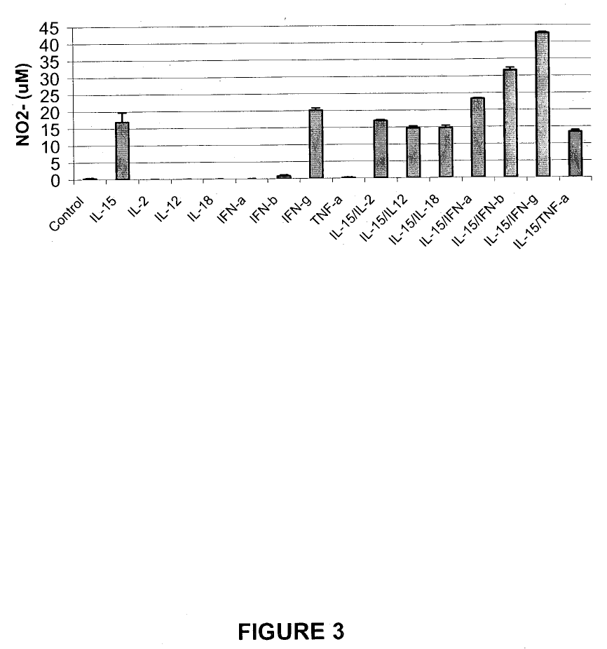

As IFN-γ has been found to induce nitric oxide-mediated inhibition of vaccinia virus replication in 293 cells by murine macrophages (Harris et al., J Virol 69:910-915), studies of nitric oxide production by the RAW 267.4 murine macrophage cell line were performed to further elucidate the mechanism of antiviral activity. IFN-γ and IL-15 increased nitric oxide production dramatically; IFN-β increased it moderately; and IL-15 enhanced the nitric oxide production induced by IFN-γ and IFN-β (FIG. 3), though no concomitant enhancement of antiviral activity was noted in the latter case.

Studies of IL-15 showed that the increase in nitric oxide production was dose-dependent. In addition a dose dependence of viral clearance for IL-15 was shown. Enhancement of nitric oxide production plays a role in the antiviral activity induced by IL-15. However, in experiments using a nitric oxide synthase blocker, the virus titer is still somewhat reduced for IL-15 in comparison with the control, indicating that another antiviral mechanism is also at work. Further preliminary experiments evaluating the ability of IL-15 to induce production of other antiviral cytokines indicate that IL-15 does not induce IFN-γ production, but does induce TNF-α production in a dose-dependent manner.

In vivo immune activation studies (without agent challenge) were performed. Cytokines (IL-2, IL-12, IL-15, IL-18, GM-CSF, TNF-α, IFN-β, IFN-γ) or cell walls administered intranasally to Balb/c mice for 5 days and spleens, peritoneal exudate cells (PECs), whole blood, and livers were harvested on day 6. The assays shown in Table 1 were performed using the harvested tissues.

TABLE 1

| Function or marker | | |
|---|---|---|
| studied | Tissue used | Assay method |
| Cytosis by NK/NKT | Splenocytes (colon-26 | Cr⁵¹ release assay |
| cells | target cells) | |
| CD69 marker | Splenocytes | Antibody staining + flow |
| expression | | cytometry |
| CD25 marker | Splenocytes | Antibody staining + flow |
| expression | | cytometry |
| Nitric oxide produc- | PECs | Greiss reaction |
| tion | | |
| Cytopathic effect of | PECs | Ceel viability assay |
| vaccinia virus on | | |
| infected KB cells | | |

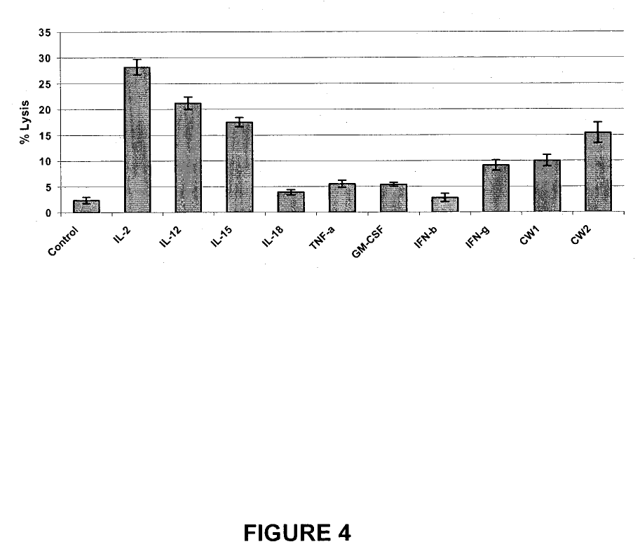

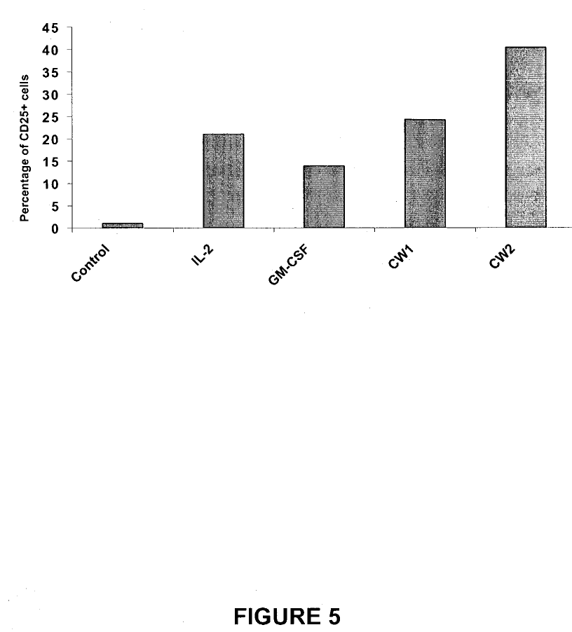

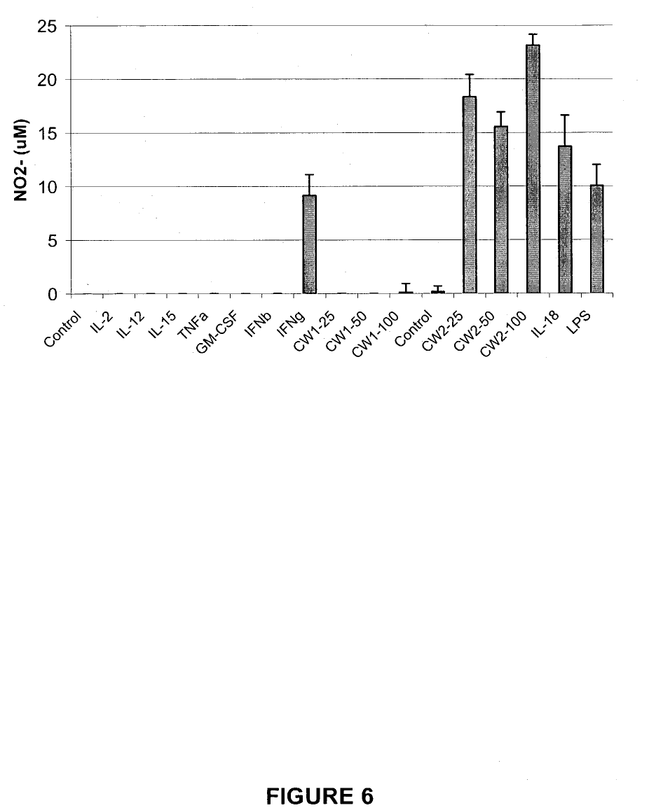

All of the animals remained healthy for the duration of the study (with one exception, a death that was determined to be unrelated to the study). In the NK/NKT studies, significant increases in cytolysis for IL-2, IL-12, and IL-15, and moderate increases for IFN-γ (FIG. 4) occurred. In the CD69 study, no CD69 expression was detected. This is most likely because CD69 is a very early activation marker, and the assay was conducted more than 20 hours after initial activation. In the CD25 marker expression study, significant activation was detected for IL-2 and GM-CSF (FIG. 5). The study of nitric oxide production revealed significant increases for IFN-γ and IL-18, but interestingly, not for IL-15 (FIG. 6), which had increased nitric oxide production in vitro. Vaccinia virus-induced cytopathic effect appeared to be reduced by PECs activated by IFN-β, IFN-γ, and IL-18 (data not shown); however, the correlation between cell viability and virus titer remains to be established.

This in vivo study demonstrated that the doses of cytokines used were well-tolerated and immune activation by the cytokines was evident, with different cytokines affecting different indicators of activation. Using these data and data from the literature, the cytokines in Table 2 were selected for use in the cytokine treatment studies using the various challenge agents:

TABLE 2

| Agent | Cytokines selected for challenge study |
|---|---|
| VEE virus | IFN-α/β, IFN-γ, IL-12, IL-18 |
| Vaccinia virus | IFN-α/β, IFN-γ, IL-15, IL-18, TNF-α |
| Yellow fever | on hold pending model development |
| virus | |
| Bacillus | IFN-γ, GM-CSF, IL-4, IL-12, IFN-α (oral, for |
| anthracis | comparison with previous literature) |
| Francisella | IFN-γ, GM-CSF, IL-4, IL-12 |
| tularensis | |

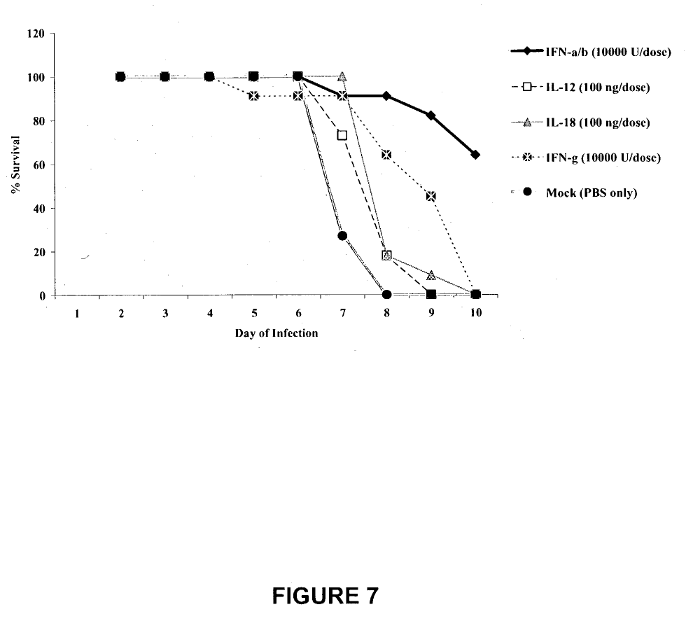

An in vivo study of cytokine treatment for VEE virus infection was also performed. Animals were treated intranasally on day −2, 0, +2, and +4 with cytokines and infected intranasally with 25 PFU of VEE virus on day 0. IFN-γ or IFN-α/β treatment increased survival of lethally VEE-infected mice (FIG. 7). IL-12 and IL-18 increased survival, but not significantly.

Blood, brain, and lungs were harvested on days 0, +2, +4, and +6 for subsequent assay of virus titer and cytokine gene expression. In general, all tissues showed some level of infection similar to mock treated animals. The various cytokines all affected virus titers, with effects varying for the different tissues. Cytokine gene expression assays and data analysis are in progress.

In the lungs, IL-12 reduced VEE titers most significantly (˜1.5 log₁₀ reduction) compared to control animals on the peak day of infection (Day+2) in the lungs. IFNα/β and IFN-γ showed less reduction (˜0.5 log₁₀), and IL-18 reduced lung titers by ˜1.0 log₁₀ on Day+2 in this tissue as well.

In blood, the levels of VEE were higher in all groups compared to controls, indicating that there was little effect of cytokine on reducing the systemic dissemination of virus from lungs once infection is established. The kinetics of viremia was different in treated animals, with peak viremia later, indicating some inhibition of dissemination kinetics perhaps by alteration of the activity or circulation of the primary target of VEE infection, the alveolar macrophage.

In brain tissues, the reduction in titer was observed only in IFNα/β treated mice. The reduction in VEE was approximately 1.25 log₁₀ compared to controls, indicating that this cytokine is useful in reducing infection in the brain. Other cytokines did not show any difference from control (untreated) animals.

Taken together, the survival and virus titer data indicate that IL-12, IL-18, IFN-α/β and IFN-γ are all good candidates. All led to some reduction in virus titer in one of three tissues examined and all had some positive effect (though not very significant) on survival after lethal challenge.

Lethality studies to establish animal models for Bacillus anthracis (Sterne strain), vaccinia virus (WR strain), and yellow fever virus (strain 17D) were conducted. LD₅₀ for mice infected intrperitoneally with Bacillus anthracis was calculated to be approximately 1×10⁶ spores; LD₈₀ was calculated to be 1×10⁷. For vaccinia virus, LD₅₀ was approximately 1.0×10⁴ PFU by the intranasal route of infection. In the lethality study using yellow fever virus strain 17D, there was no disease or lethality at any infectious dose.

IL-15-induced NO production and antiviral activity are well correlated. Both NO production and vaccinia virus clearance exhibit dose dependence on IL-15. Therefore IL-15-induced antiviral activity is mediated mostly by NO production.

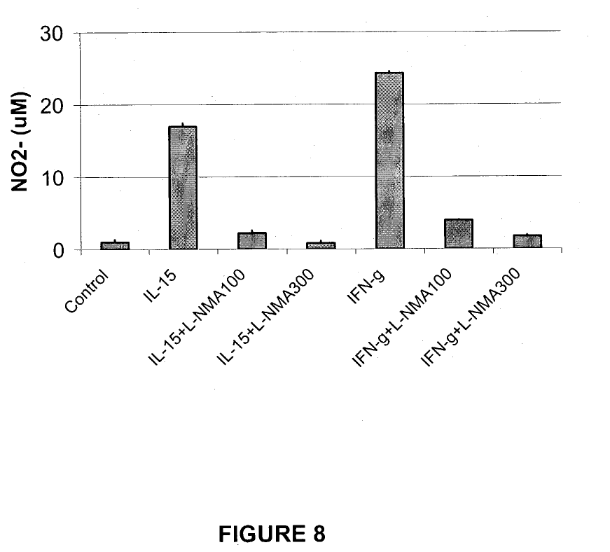

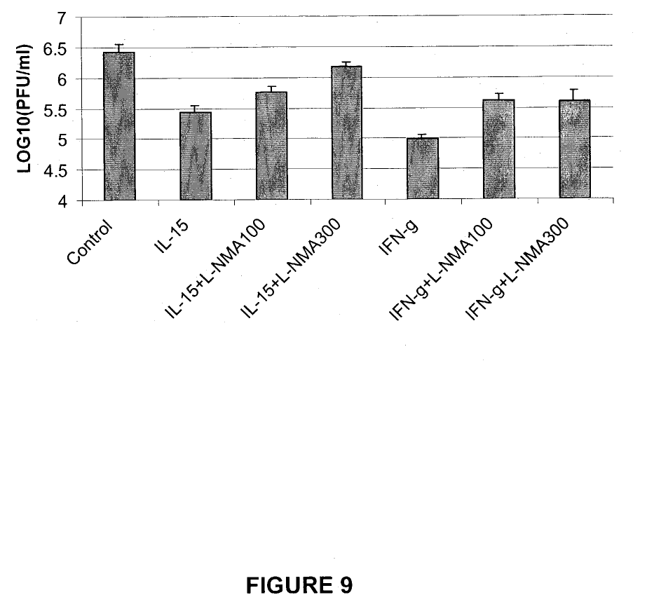

To confirm the role of NO in antiviral activity, the effects of a nitric oxide synthesis inhibitor, N^{G}-monomethyl-L-arginine acetate (L-NMA), on NO production and vaccinia virus clearance was studied. L-NMA was found to inhibit NO production and restored VV replication (see FIGS. 8 and 9).

No IFN-γ above 128 pg/ml was detected in IL-15-activated RAW cells by ELISA. Neutralizing anti-IFN-γ monoclonal antibody significantly blocked IFN-induced NO production and restored vaccinia virus replication. This antibody did not block IL-15-mediated NO production, suggesting that IL-15 induces NO production directly or through another mechanism that does not involve IFN-γ. In order to demonstrate conclusively that the IFN-γ-induced pathway for NO production plays no role in IL-15 induction of NO synthesis, mRNA synthesis of inducible nitric oxide synthase (iNOS) and IFN-γ genes can be quantified by RT-PCR in IL-15 activated macrophages. This can also be done to test NO production by RAW 264.7 gamma NO(−) cells.

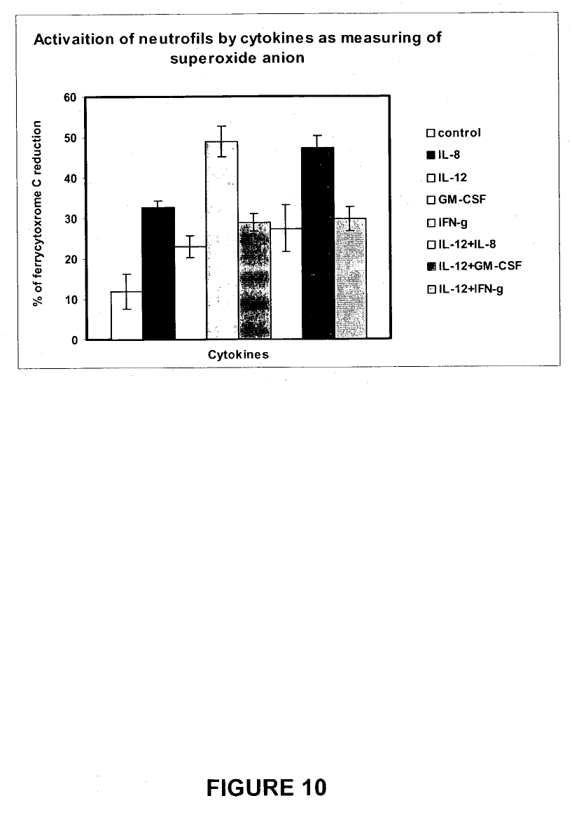

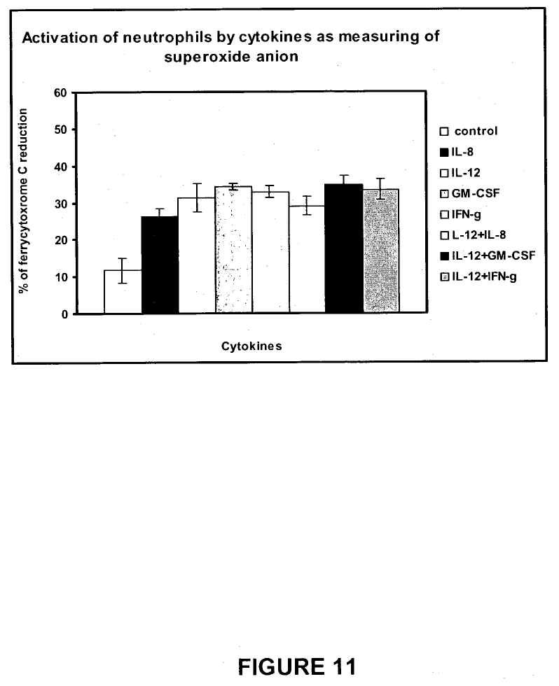

Studies on the effects of cytokines on human neutrophil function as measured by superoxide anion production were performed. All of the cytokines tested (IL-8, IL-12, GM-CSF, IFN-γ) and their combinations increased superoxide anion levels. The level of increase was donor-dependent. (FIGS. 10 and 11)

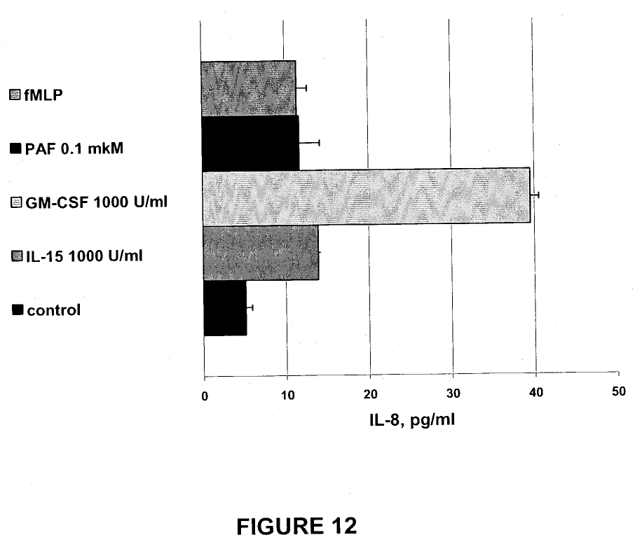

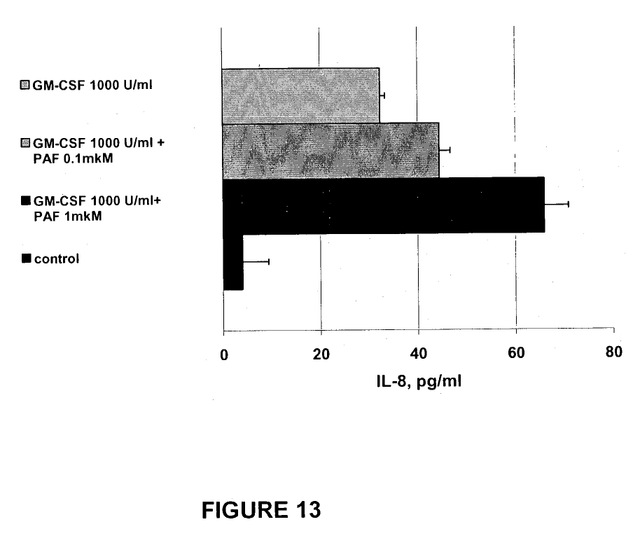

The effects of cytokines on IL-8 production by neutrophils were studied. Granulocyte-macrophage colony stimulating factor (GM-CSF) significantly increased IL-8 production by neutrophils, while IL-15 and platelet activating factor (PAF) increased IL-8 production moderately (FIG. 12). Additionally, PAF enhances GM-CSF-induced IL-8 production (FIG. 13).

A high-performance liquid chromatography (HPLC) system from ESA, Inc. with a detector array of sixteen programmable electrodes was used to determine the effect of cytokines and cell walls on the tryptophan metabolic pathway of immunocompetent cells. This system detects on the order of 2000 analytes under optimal conditions. Experiments to designed to maximize detection and quantitation of tryptophan metabolites can be performed.

A previously unknown tryptophan-like species was detected in extracts from IFN-γ-treated murine macrophages. There are unique changes in analyte profiles of LPS-treated versus untreated mouse macrophage cells. The observed alterations in analytes associated with both the LPS and INF-γ treatments are observed prior to any detectable iNOS activity.

Two components were identified in this study. Component A is a component of unknown structure, with a retention time of 4.54 minutes, was detected in channels 8-9 and was present in controls and retained on treatment of cells with LPS alone. However, it was removed in macrophages treated with INF-γ alone or in combination with LPS. Experiments can be done to obtain sufficient materials from additional treatments to obtain high performance liquid chromatography—mass spectrometry (HPLC/MS) data on this analyte to determine its structure.

Component B, while present in macrophage controls, showed significant alterations in concentrations upon treatment of macrophages with both LPS and INF-γ. Component B has a retention time of 22 minutes and appears just prior to tryptophan in our chromatographic system. It was detected within channels 10-12, as is tryptophan, and appears from its electrochemical and chromatographic behavior to be an indole derivative. This component in controls was less than 10% of the intensity of tryptophan. Treatment with both LPS and INF-γ decreased the levels of detectable tryptophan, while increasing the amounts of component B. The effect was enhanced when cells are treated simultaneously with INF-γ and LPS.

The data set from these assays can be converted into formats allowing both cluster analysis and PCA (principal component analysis) to expand the information extractable from the data and its relevance to specific effector agents. Complete data analysis on quantitative changes throughout the analyte profiles can be developed.

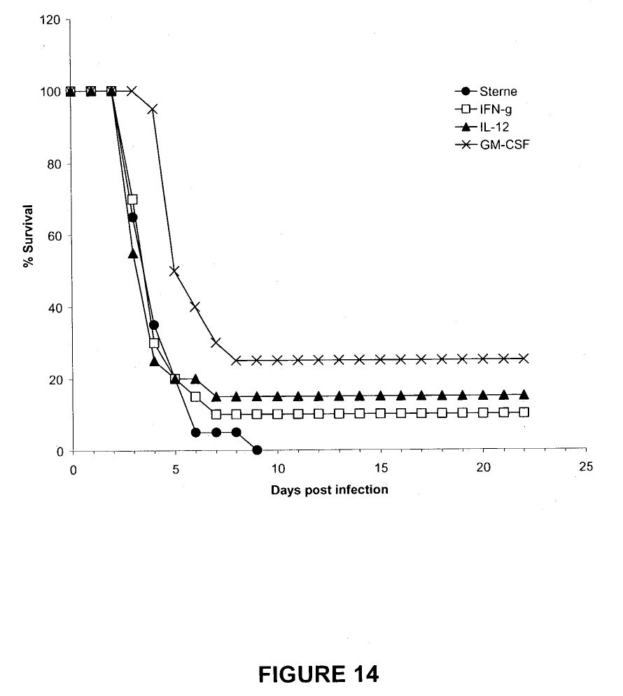

In vivo studies of the protective effects of cytokines against agent challenge such as Bacillus anthracis and vaccinia virus were performed. In the anthrax study, IL-12, IFN-g, and GM-CSF all increased survival (mice still alive at day 23, study ongoing), with GM-CSF exhibiting the greatest effects (see FIG. 14). Granulocyte colony-stimulating factor (G-CSF) can be added in these studies. In this study, spleens were harvested on days +2, +4, and +6 after infection for determination of bacterial load; no correlation of bacterial load with survival was observed.

IL-15, IL-18, and TNF-α all appear to have a negative effect, as some of the animals in these groups died.

IFN-γ and IFN-α/β increased survival Venezuelan equine encephalomyelitis (VEE) virus. RNA was obtained from the spleens harvested during this study, and can be used for cytokine gene expression studies during the next quarter. Studies with IFN-α/β administered intranasally can also be performed. In addition, the effect of different administration routes using three different doses of oral IFN-α (1 U, 10 U, 100 U) can be performed.

Computer Modeling of Cytokine-Receptor Interactions for Rational Design of Peptide/Small-Molecule Mimetics

Dynamic models of receptor-ligand interaction can be created. The ProMax™ TNF module can be used for this purpose.

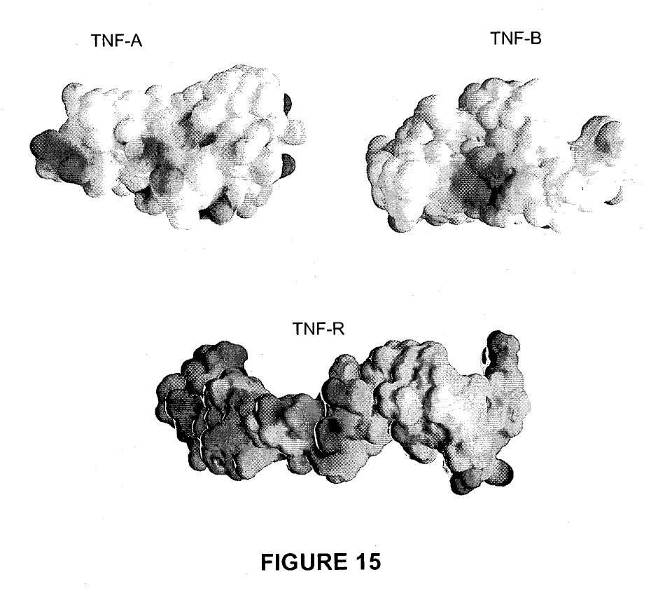

Structures for IFN-α, IFN-γ, IL-10, and IL-15 have been generated and added to a database. In addition, dynamic simulation trajectories for cytokines in the database were completed. These molecular dynamics simulations were conducted for 500 picoseconds. Analysis of the specific trajectories was conducted to provide the basis for initiating the modeling of ligand-receptor complexes. FIG. 15 shows typical results for TNF-A, TNF-B, and TNF-R, where the red shading indicates regions exhibiting high structural flexibility, white indicates low flexibility, and blue indicates moderate flexibility.

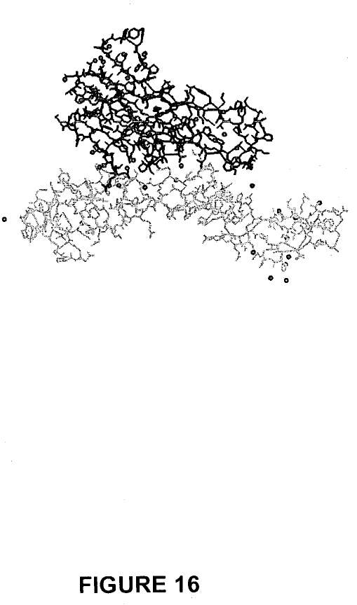

Modeling of cytokine ligand-receptor complexes can also be performed. This activity can provide the basis from which the mimetics can be designed. A complex model can be made for TNF-A and TNF-R. A docked complex structural model was designed and then refined using energy minimization followed by 500 picoseconds of solvated molecular dynamics. The simulation was successful, resulting in a stable model. FIG. 16 presents a view of the structural model of the TNF-A-TNF-R complex.

Dynamic modeling of TNF-α and TNF-β of ligand-receptor complexes was performed. This provided the data to characterize the structural geometries of the pharmacophoric regions on both TNF-α and TNF-β for subsequent use in designing the candidate mimetics.

The protocol used for the design of peptide mimetics for TNF-α and TNF-β. is as follows:

Imitate parts of TNF surfaces so as to agonize some positive functions of TNF such as stimulation of neutrophils for oxygen radical production, degranulation, and associated microbicidal and tumoricidal activities, while not triggering the toxic effects of TNF.

Relatively low molecular weights.

Design cyclization to further stabilize the “native” folds of peptide mimetics.

Energy minimize the peptide mimetics to achieve reasonable geometry.

Run solution molecular dynamics simulation to examine the conformational stability of the peptide mimetics.

Compare the similarity between the peptide mimetics and the patches of TNF-a or b in terms of the solvent accessible surface, hydrophobicity distribution and electrostatic distribution.

A set of small molecules that bind to TNF receptor (TNFR) by screening the pharmacophores against its small molecule virtual library can be used for in vitro screening for toxicity and ability to block TNFR.

Small-Molecule Innate Immunoligands (SMILS) as Antimicrobial Agents

Cell walls from 40 Gram-positive microorganisms were isolated. Cytokine production was evaluated for GM-CSF, IL-10, TNF-α, IFN-γ, IL-12, IL-1β and IL-6. The cytokine and nitric oxide release studies in murine macrophages stimulated with cell wall preparations indicated that the different bacterial cell walls induced release of cytokines and reactive nitrogen species at varying levels, with substantial differences in cytokine release profiles among the various cell walls tested.

Fractionation techniques were used to successfully fractionated cell walls from five Gram-positive bacteria. Studies of cytokine release and nitric oxide production stimulated in macrophages by the various cell wall components can be performed. In vivo studies of cell wall activation can be performed by administering two different bacterial cell walls.

Cytokine gene expression by macrophages in response to stimulation by B. anthracis cell wall (CW) was evaluated. Gene expression was followed by isolating mRNA from a cell wall-stimulated human THP-1 cell line, performing RT-PCR, and then attempting multiplex PCR of various cytokine genes. THP-1 cells grown continuously in cell culture were stimulated by addition of either 1 μg of CW or 0.1 μg of pokeweed mitogen and then incubated for 2 hours. Cytokines and chemokines tested were: IL-1β, IL-2, IL-4, IL-5, IL-6, IL-8, IL-10, IL-12, IL-13, IP-10, TNF-α, TGF-β, MCP-1, MCP-2, INF-γ, GM-CSF, RANTES, and GADPH. GADPH was typically used as a housekeeping gene and as a measure of cell and preparation viability.

Results of the tests indicated increased expression of IL-8 upon exposure to CW. Furthermore, incubation of THP-1 cells with mitogen, as well as dual incubation with both mitogen and CW, yielded induction of IL-8 transcription. IL-12 and IL-13 were both detected upon CW stimulation. Significant MCP-2 and IFN-γ signal was detected in cells stimulated with CW. RANTES and GADPH expression could be followed throughout all samples, indicating cell viability throughout the assay.

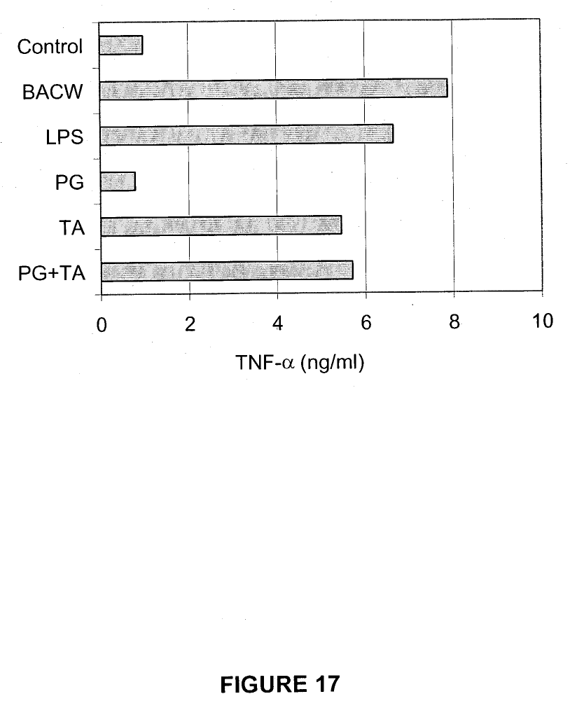

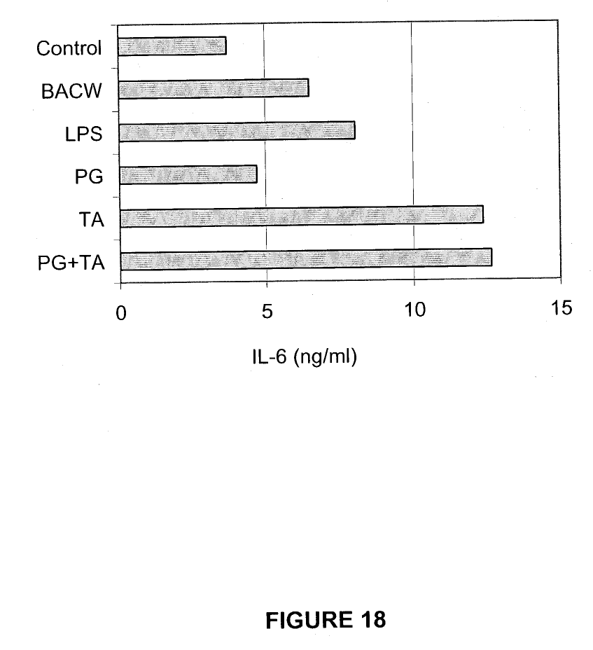

Studies of the cytokine release and nitric oxide production of murine peritoneal macrophages (PECs) were performed. The PECs were activated with IFN-γ for 24 hours and stimulated with Bacillus anthracis cell wall (BACW, 1 μg/ml)) or its components, peptidoglycan (PG, 10 μg/ml) and/or teichoic acid (TA, 10 μg/ml) for 48 hours. Lipopolysaccharide (LPS)-stimulated cultures were used as a positive control. Release of interleukin-1β, IL-6, TNF-α, and IFN-γ was detected by ELISA. Stimulation of murine PECs from DBA/2J strain and CBA/J strain (data not shown) led to increased levels of IL-6 and TNF-α (FIGS. 17 and 18). We also observed elevated levels of IL-1β and IFN-γ (data not shown). Stimulation with PG alone did not lead to increased cytokine production, but stimulation with TA did. There were no differences in cytokine response between the two murine strains.

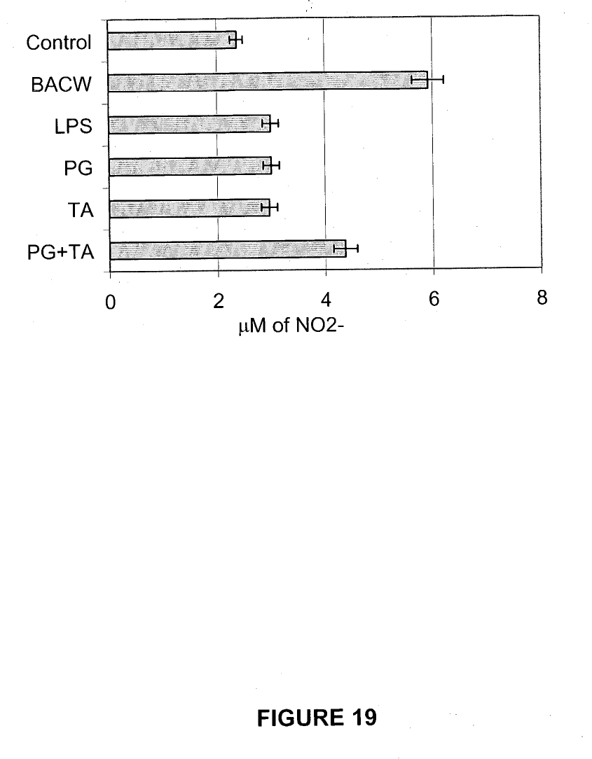

Nitric oxide production by PECs from BALB/c mice treated as described above with IFN-γ and BACW, PG, TA, PG+TA, or LPS was measured. The concentration of nitric oxide in the samples was measured indirectly as nitrite in the media using Greiss reagent. Absorbance was determined at 540 nm and NO was calculated in limoles using a calibration curve of sodium nitrite solution. These studies showed that BACW stimulation resulted in a 2.5-fold increase in production of NO by murine PECs. Stimulation with PG or TA alone resulted in no increase in NO production, while the increase observed for PG+TA was not significant (FIG. 19).

Eighty-four purified crude cell walls, 66 from various Gram-positive bacteria and 18 from yeast, were prepared. Twenty-seven bacterial cell walls have been fractionated for peptidoglycan and 23 for teichoic acid. Fractionation for glucan is in progress for all 18 yeast cell walls. Further fractionations of cell wall components to lipoproteins, lipoteichoic acids, and mannoproteins can be performed.

In vitro studies of cytokine release and nitric oxide production by cell wall-stimulated murine peritoneal macrophages for an additional 10 crude cell walls (beyond the initial 15 previously completed) were performed. The results indicate that different cell walls and their components induce cytokine release and reactive nitrogen species production at different levels. In addition, IFN-γ increased cell wall-induced nitric oxide production by 10-20 μM. The cell walls that induces the greatest increases in nitric oxide production can be assayed for their ability to affect the antiviral activity of macrophages in vitro.

Cytokine gene expression studies, previously conducted, were conducted on murine PECs. The results showed that there was increased expression of IL-6, IL-1μ, TNF-α, IFN-γ, and IL-13 in Balb/c mice exposed to B. anthracis cell wall or LPS. For A/J mice, increased expression of IL-6, IL-1β, and TNFα. TGF-β was continuously demonstrated throughout the controls and exposed cells due to serum in the media.

In vivo studies of immune activation by two crude cell wall preparations, from Bacillus anthracis (CW1) and Staphylococcus caseolyticus (CW2), were performed. Cell wall preparations (25, 50, or 100 μg of either CW1 or CW2) were administered intraperitoneally to a group of mice daily for 5 days, with LPS (25 μg) serving as a positive control. Tissues were harvested on day 6 and the following studies shown in Table 3 were conducted:

TABLE 3

| Function or marker | | |
|---|---|---|
| studied | Tissue used | Assay method |
| Cutolysis by NK/NKT | Splenocytes (colon-26 | Cr⁵¹ release assay |
| cells | target cells) | |
| CD69 marker | Splenocytes | Antibody staining + flow |
| expression | | cytometry |
| CD25 marker | Splenocytes | Antibody staining + flow |
| expression | | cytometry |
| Nitric oxide produc- | PECs | Greiss reaction |
| tion | | |
| Cytopathic effect of | PECs | Cell viability assay |
| vaccinia virus on | | |
| infected KB cells | | |
| Cytokine production | Plasma (from whole | ELISA |
| | blood) | |
| Cytokine gene | Liver tissue | RT-PCR |
| expression | | |

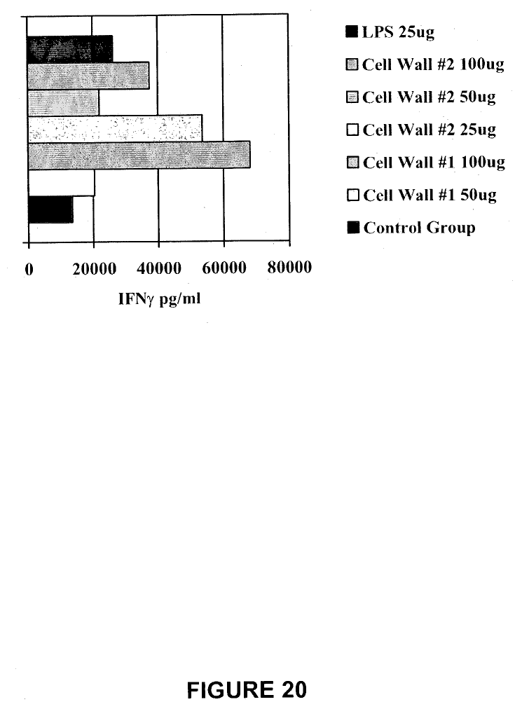

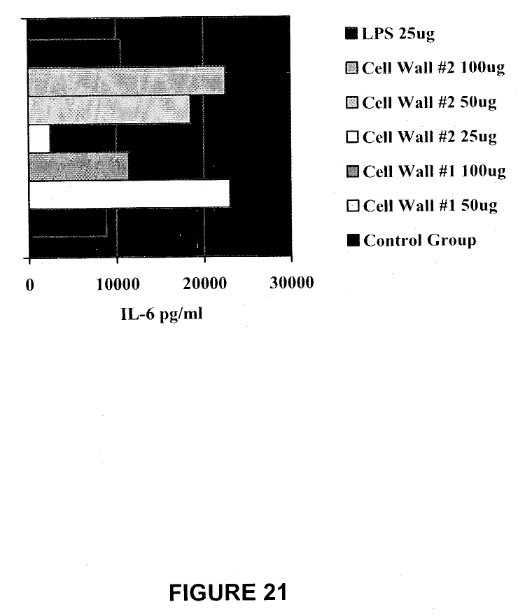

Animals in the CW2 and LPS groups were ill, but still living, at the end of the study; the remaining animals were healthy. In the NK/NKT cytolysis study, significant increases in cytolysis were observed for CW2 and moderate increases for CW1 (See FIG. 4). In the marker expression studies, again, no CD69 expression was observed. However, significant activation by both cell wall preparations was detected in the CD25 study (see FIG. 5). CW2 elicited significant increases in nitric oxide production by splenocytes (see FIG. 6). Studies of the production of IL-1β, TNF-α, IFN-γ, and IL-6 indicated that the cell walls generally increased production of those cytokines. Again, the different cell walls had different effects; the increases in IFN-γ and IL-6 were the most varied (see FIGS. 20 and 21; samples for the 25 μg dose of CW1 were not available). In the cytokine gene expression studies, livers from mice challenged with CW1 showed increased IL-1β expression over controls, but no other cytokines were detected.

These two cell wall preparations indeed activate various elements of the immune system, and that cell wall preparations in general can be studied. The top cell wall candidates that emerge from in vitro studies in a similar in vivo activation study can be evaluated, and if any candidates are promising, their antiviral effects against pathogen challenge can be evaluated.

Studies of effects of cytokine and cell wall stimulation on the tryptophan metabolic pathways of immunocompetent cells were performed. IFN-γ-treated macrophages and fibroblasts degrade the amino acid tryptophan into a series of well-characterized degradation products. Among these are quinolinic and picolinic acids. These compounds arrest growth of bacteria in vitro, suggesting a role in host defense; their activities are distinct from the known oxygen or nitrogen metabolites synthesized during macrophage activation. An unknown tryptophan-like species was identified in extracts from IFN-γ-treated murine macrophages. Studies to determine whether it possesses antimicrobial activity in vitro, then in vivo can be performed. Studies on the effects of selected cell walls on tryptophan metabolic pathways can be performed.

Of the 84 bacterial and fungal cell walls isolated previously, an additional seven were fractionated for peptidoglycan and teichoic acid. In addition, the techniques to fractionate for lipoteichoic acid were determined. Fast-performance liquid chromatography (FPLC) can be used in fractionation. The protein content in the crude cell walls was characterized and screened for protein contamination in the peptidoglycan and teichoic acid fractions. The inorganic phosphate and DNA contamination in the crude cell walls and fractions can be measured.

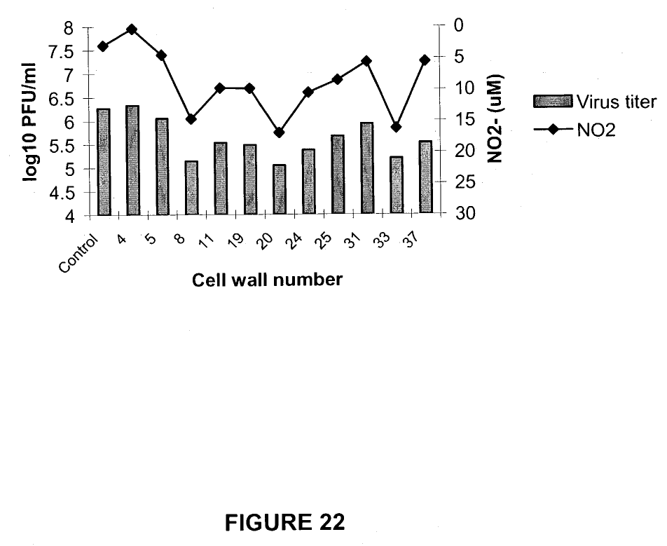

In vitro studies of the effects of crude cell walls included assays of Vaccinia virus clearance by murine macrophages. Thirty-nine crude cell walls were tested for antiviral and NO activity. Some cell walls increased NO production in murine macrophages, and the ten with greatest increases for studies of vaccinia virus clearance by the macrophages were selected. NO production and virus clearance were well-correlated (FIG. 22). In addition, combinations of cell wall and cytokine (IL-15 or IFN-γ) on vaccinia virus clearance were tested. IL-15 had no effect on cell wall-enhanced clearance, but there was a possible weak synergetic effect of IFN-γ.

Superoxide anion production by human monocytes and neutrophils was studied. The study of cell wall effects on superoxide anion production by human monocytes showed that most cell walls activate superoxide anion production, some activate it, and some have no effect. Significant donor variability was noted.

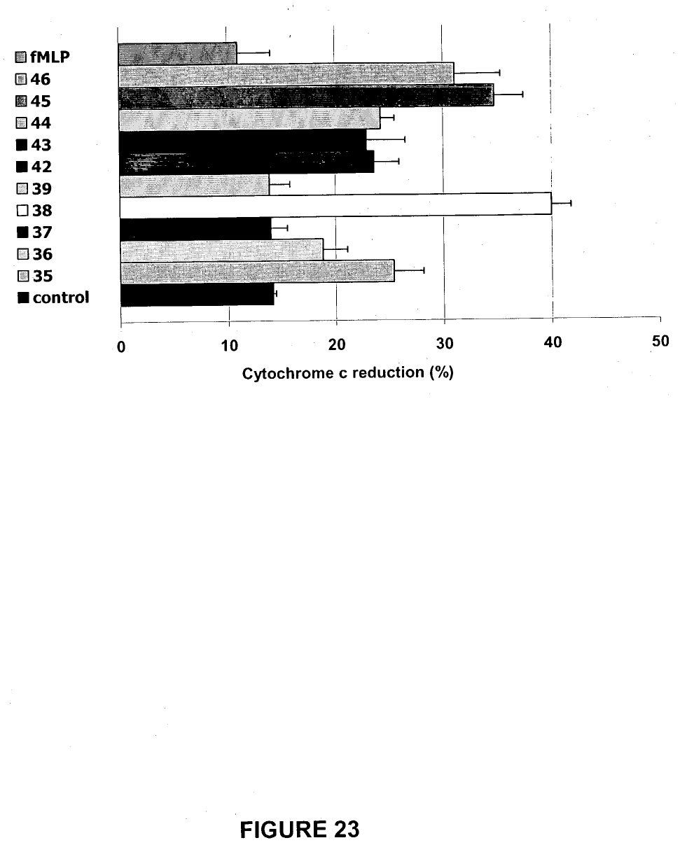

In studies on superoxide anion production by neutrophils, about two-thirds of the cell walls tested increased superoxide anion production (FIG. 23). Again, significant donor variability was noted.

Cytotoxic activity of human NK cells (PBMC) against target cells was studied. Most of the cell walls tested enhanced the cytotoxic activity of PBMC against target cells (K562). This was demonstrated by the MTT assay method described previously and confirmed by the Cr⁵¹ release assay method. In addition, none of the cell walls was found to be inherently cytotoxic.

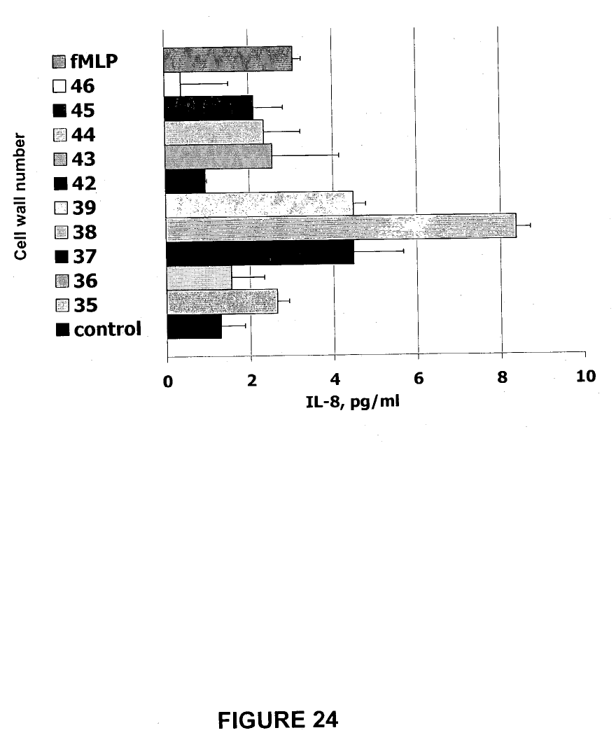

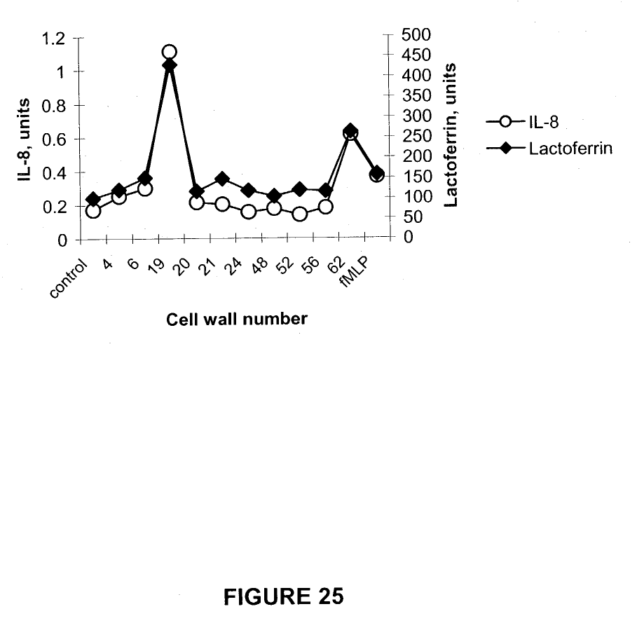

Release of IL-8, lactoferrin, surface L-selectin by neutrophils was also studied. In neutrophils activated with cell walls, about one-third of the cell walls tested increased IL-8 production (FIG. 24). Lactoferrin production increased similarly to IL-8 production (FIG. 25). Several cell walls also decreased surface L-selectin.

Chemokine gene expression of human PBMC was also studied. In a preliminary study on human PBMC stimulated with B. anthracis cell wall, RNA was isolated using the TriZol method and gene expression was determined using the RNAse protection assay. This cell wall stimulated production of Mip-1α, Mip-1β, IL-1α, and IL-1β, as well as IL-8, as previously reported.

The ten most promising cell walls can be tested for expression of activation markers and release of cytokines/chemokines.

In an immune activation study, the data obtained for three doses each of two cell walls provides sufficient information to select a dosage for further in vivo studies. All of the doses studied activated various elements of the immune system, and none of the animals in the study died. Therefore the lowest dose tested (25 μg/mouse/day) can be used.

In vivo studies of three cell walls against anthrax challenge and VEE challenge can be performed. These studies can be conducted together with the cytokine treatment studies for the same infectious agents. The in vitro data can be used to select additional cell walls for further in vivo studies.

Compilation of a database on the effects of the various cell walls on immunocompetent cells in the in vitro studies as well as in the in vivo studies can be performed.

According to current theories, LeTx plays a major role in the development of anthrax sepsis, septic shock, and death. However, anthrax bacilli accumulate in the bloodstream at a logarithmic rate, with little bacterial clearance by the immune system. Continued bacterial proliferation leads to a significant decrease in host oxygen and nutrient availability, with a concurrent increase in both bacterial and host metabolic waste products. As the available oxygen is used, the bacteria reach a stationary growth rate. Overproduction of proinflammatory mediators, coupled with severe hypoxia from the consumption of oxygen by proliferating bacilli, leads to organ failure, the development of shock and sudden death.

The invention provides for a combination therapy for anthrax which includes both antibiotics and antibodies.

The invention provides for administration of antibodies. In one embodiment the amount of antibody administered to the patient is from 100 mg to 400 mg. In another embodiment, the amount of antibody is from 120 to 360 mg. In another embodiment, the amount of antibody is from 180 to 300 mg. In yet another embodiment the amount of antibody is from 200 to 280 mg.

The amount of antibody can also be determined on a per weight basis. Doses of antibodies range of the invention range from 0.1 mg/kg to 100 mg/kg or more. Other embodiments include doses of 1 mg/kg to 50 mg/kg. In yet other embodiments the amount of antibody is from 5 mg/kg to 25 mg/kg. Preferably, 10 mg/kg of antibody is administered.

The invention also provides for the administration of antibiotics. The antibiotic ciprofloxacin is administered either orally or intravenously. Oral administration can be at a dose of from 100 mg to 750 mg, including 250 mg, and 500 mg, every twelve hours. In children, oral administration of ciprofloxacin is 15 mg/kg per dose up to 500 mg per dose. Intravenous ciprofloxacin is administered every twelve hours in doses ranging from 200 to 400 mg. In children ciprofloxacin is administered intravenously at 10 mg/kg up to 400 mg per dose. Treatment with ciprofloxacin lasts from 5 to 60 days.

In an embodiment of the invention, antibodies will be administered intravenously or subcutaneously, and antibiotics will be administered perorally, intravenously, or subcutaneously. Injectable forms of the antibiotics or antibodies can be administered intravenously or subcutaneously, while peroral administration can be achieved by many different methods, including but not limited to, with tablets, solutions lozenges, etc.

The antibodies of the invention can be polyclonal or monoclonal. Monoclonal antibodies can be prepared as described by Kohler and Milstein (1975). In an embodiment of the invention, monoclonal antibodies can be engineered to be chimeric antibodies, including human constant regions. The antibodies of the invention may be raised in any species of animal, including but not limited to, rabbits, sheep, horses, mice, goats, monkeys, rats, etc. Sheep antibodies are the preferred embodiment.

In embodiments of the invention, the antibiotics include, but are not limited to fluoroqinalones, tetracyclines, β lactams or other antibiotics recommended for treatment of anthrax. Ciprofloxacin and doxcycline are the preferred embodiments.

In one embodiment of the invention the antibiotic and the antibodies are administered at the same time. In another embodiment, the antibiotic and antibodies are administered serially, with either the antibody or the antibiotic administered first.

In yet further embodiments of the invention, other blockers of anthrax toxin action, such as molecules that prevent anthrax toxins from binding to and entering human cells are provided in addition to the antibiotic and antibody treatments of the invention.

The invention provides for treatments, both therapeutic and prophylactic, for smallpox infection and anthrax infection. These treatments are administered as an inhaled preparation to the infected individual. In one embodiment of the invention, a combined preparation of cytokines and bacterial cell walls is indicated. This embodiment encompasses cytokines including INF-α, IFN-γ, and GM-CSF. This embodiment also encompasses cell wall of the bacteria B. alcalophilus, E. faecium, S. caseolyticus, and B. stearothermoohilus. The peptidoglycan fraction of B. alcalophilus cell wall can be used in the combined treatment preparation.

In addition, the invention provides for inhaled treatments for anthrax and smallpox infection comprising individual components. INF-α, IFN-γ, GM-CSF, and the cell walls of B. alcalophilus, E. faecium, S. caseolyticus, and B. stearothermoohilus, as well as the peptidoglycan, lipoteichoic acid, and muramyl peptide fraction of these cell walls can be used as treatments, both therapeutic and prophylactic, for smallpox and anthrax.

Embodiments including muramyl peptides provide induction of a broad spectrum of cytokines that affect immune cells, such as, but not limited to, macrophages, NK/NKT cells, and T cells. Muramyl peptides can also be provided at relatively low cost and can be administered easily by oral routes. Finally, macrophage activation after exposure to muramyl peptides is rapid, occurring within a few hours.

Treatments with these cytokines and cell walls is provided at doses which are lower than those that cause toxic effects in humans. IFN-α has been administered at up to 300 million IU/m² subcutaneously, without adverse reactions. IFN-α is typically administered at 2-20 million IU/m², either intravenously, intramuscularly, or subcutaneously. Treatments with INF-α at these doses can last for four weeks to 6 months or a year. INF-γ is typically administered at 150 μg by subcutaneous injections. This treatment can typically last for 4 weeks or longer. Recombinant INF-γ can also be administered at doses of 0.01 to 2.5 mg/m² by alternating intramuscular and intravenous bolus injections with a minimum intervening period of 72 h (Kurzrock et al. 1985). Recombinant GM-CSF is typically administered intravenously, most preferentially at 6.25 μg/kg/day over a four hour period. Doses up to 100 μg/kg/day of GM-CSF can be administered. As known by those skilled in the art, administration of cytokines by inhalation, as provided for in the invention, will require significantly lower doses than those recited here by injection. These lower doses will reduce any adverse side effects of these cytokines.

Cell wall preparations have been administered in several different ways. Oral preparations can be taken with meals three times a day at 3-6 g per dose (Krusteva et al. 1997). Cell wall has also been administered by pleural injection of 1 mg of dried bacteria, once a week for four weeks (Luh et al. 1992). In other settings, cell wall has been administered intradermally at a dose of 0.1 mg twice a week or 0.2 mg once a week. Longer treatments have been done for up to two years at 0.1 mg every two weeks or 0.2 mg every month (Okawa et al. 1993). Administration of cell wall by inhalation is known to those skilled in the art to require lower doses.

To achieve prophylaxis, the administration of treatments of the invention, including combined cytokine and cell wall treatments, can be administered before infection. In an embodiment of the invention, prophylaxis can be achieved by administering combined cytokine and cell wall therapies at least twice before infection.

In a further embodiment of the invention, the prophylactic and therapeutic treatments of the invention can be used for individuals at risk or high risk for adverse effects of the smallpox vaccine currently available, including but not limited to those with deficient immune systems or suffering from eczema.

In yet another embodiment of the invention, the invention provides for both therapeutic and prophylactic treatments for other poxvirus infections, such as human monkeypox. Other poxvirus infections include, but are not limited to, molluscum contagiocum virus (MCV), which is a world wide opportunistic infection among AIDS patients.

The compositions of the invention can be incorporated into liposomes or can be micorencapsulated for administration to a patient. Other methods of stabilizing the compositions in the blood can also be used in the invention.

In one embodiment, the therapy comprises a composition of cytokines and optionally bacterial cell walls or components of bacterial cell walls.

In embodiments of the invention, the cytokines that can be included in the composition of the therapy include, but are not limited to, IFN-α and IFN-γ.

In embodiments of the invention, bacterial cell wall or fractions of bacterial cell wall are included in the composition of the invention. The fractions of the cell wall include, but are not limited to, peptidoglycan fractions.

A combination of IFN-α and IFN-γ are the preferred embodiments for treatment of poxvirus, including smallpox, infections. Furthermore, a combination of IFN-γ and the peptidoglycan fraction of B. alcalophilus is also preferred for treatment of poxvirus infections. A combination of GM-CSF and the cell wall of cell wall components of B. alcalophilus are the preferred embodiments for treatment of anthrax.

Cytokines are preferably administered intranasally as a therapeutic composition. Other routes of administration include oral, subcutaneous, intramuscular, intravenous, or intraperitoneal.

In one embodiment the composition of the invention can be used for pre-exposure prophylaxis. In another embodiment of the invention the composition is used for post-exposure treatment.

This invention will be described in greater detail in the following Examples:

### EXAMPLE 1

Antibody-Based Treatment for Anthrax Infection

Testing of antibodies as inhibitors of anthrax lethal toxin-induced cell death was performed on the toxin-sensitive murine macrophage-like cell line RAW 264.7. Briefly, different concentrations of the antibodies were pre-incubated with and without LeTx for 1 h in a CO₂ incubator, then added to RAW 264.7 cells and incubated for 4 h in a CO₂ incubator. Cell death was monitored by MTS-based colorimetric viability assay. Monoclonal antibodies against PA were obtained from Biodesign International (catalog # C86613M). Polyclonal antibodies against heat-inactivated vegetative Bacillus anthracis (Sterne strain), bacterial cell wall, and PA were produced by immunization of rabbits (performed by Spring Valley Laboratories, Inc., CO) using antigens provided by Advanced Biosystems, Inc.

Briefly, a 52-day protocol was used to obtain polyclonal antibodies. On day 0 a pre-immunization bleed was taken and primary immunization with PA was preformed subcutaneously with an Freund's complete adjuvant emulsion. On day 21, an immunogen boost with PA was performed subcutaneously with an Freund's incomplete adjuvant (FIA) emulsion. On day 31, a bleed was taken and ELISA analysis performed. On day 42, another immunogen boost was performed subcutaneously with an FIA emulsion preparation of PA. On day 52 a bleed was taken and according to the titer obtained by an ELISA assay, exsanguination was performed

Polyclonal antibodies were received in the form of total IgG isolated from immune plasma and were characterized by ELISA.

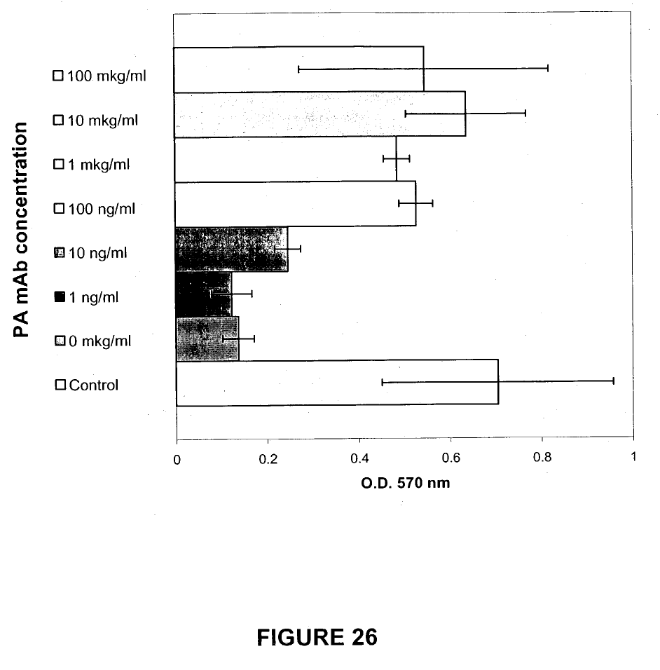

Antibodies as inhibitors of lethal toxin-induced cell death. The ability of specific antibodies to inhibit the cytotoxic activity of lethal toxin was evaluated by incubating of RAW 264.7 cells with variable concentrations of the antibodies in the presence or absence of LeTx. When the monoclonal antibody against PA was used, complete protection of the cells was achieved at 100 ng/ml of monoclonal antibody (FIG. 26). Protection was dose-dependent. From a drug discovery standpoint, effects observed at less than 1 μM concentrations are generally considered significant. In these experiments nanomolar concentrations were used. No cytotoxic effect of antibody alone was noted across the range of concentrations used.

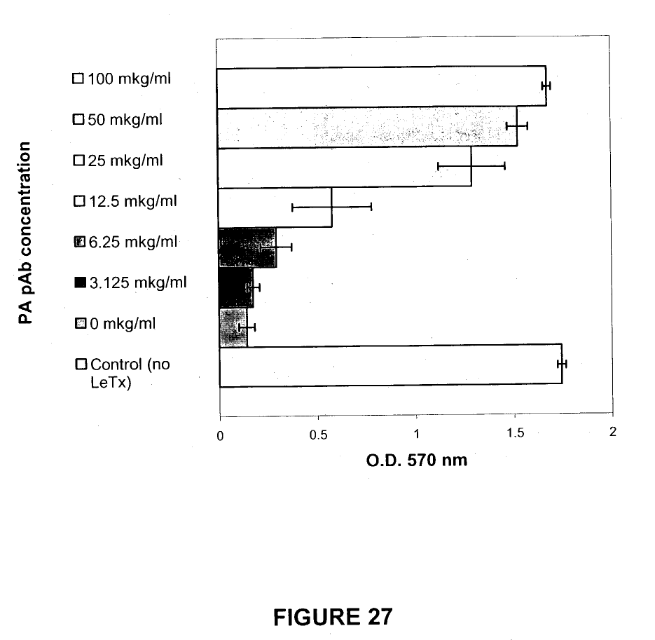

Polyclonal antibodies against PA, in the form of total IgG isolated from immune plasma, also demonstrated protective properties but at much higher concentrations (FIG. 27). Anti-PA antibodies can inhibit LeTx action. Neutralizing monoclonal antibodies, depleted polyclonal IgG can also inhibit LeTx.

IgG preparations from polyclonal antibodies against heat-inactivated vegetative B. anthracis (Sterne) bacteria and against a preparation of B. anthracis cell wall were obtained. The antibodies against the inactivated bacteria were tested for their ability to inhibit lethal toxin-induced cytolysis in order to determine whether PA and/or lethal factor (LF) were present among the other bacterial components inducing antibody production. This IgG did not display protective properties even at the highest concentration tested, 100 μg/ml. Thus, while antibodies to PA or LF may be present in this IgG sample, their concentration is too low to provide significant protection against LeTx-induced cell death.

### EXAMPLE 2

Combination Therapy of Antibodies and Antibiotic

The combination of ciprofloxacin with IgG containing polyclonal antibodies against heat-inactivated vegetative B. anthracis (Sterne) bacteria or/and against PA were tested for their ability to protect DBA mice against anthrax infection.

Antibodies against heat-inactivated vegetative B. anthracis (Sterne) bacteria were obtained by using heat inactivated bacteria as an antigen to raise antibodies as described above. B. anthracis Sterne Strain was obtain from the Colorado Serum Company (4950 York Street, Denver, Colo. 80216 and was originally developed at the Onderstepoort Laboratory, Pretoria, South Africa. A beef heart was prepared for infusion. An aliquot of B. anthracis frozen (0.5-1-mL) was added to 100-mL of the supplemented beef heart for infusion. The culture was then Incubated for 4 hours at 37° C. to a cell density of 109 cells/mL in broth media. Optical density measurements were taken hourly, until the optimal density was between 0.6-1.0 for vegetative cells. Cultures were then sedimented at 8000 RPM for 15 minutes at 4° C. in centrifuge tubes. The culture medium was removed and re-suspended in 50 mL of PBS. Tubes containing the culture were boiled in water for 30 minutes. To test viability of cells, 10 μL of boiled bacterial suspension was added to 10 mL of beef heart for infusion broth in 15 mL conical tube. The tubes were incubated at 37° C. for 24-48 hours and checked for turbidity. To lyophilize the sample, the culture were sedimented and resuspend in 3-5 mL of PBS. The samples were aliquoted and lyophilize for 24 hours.

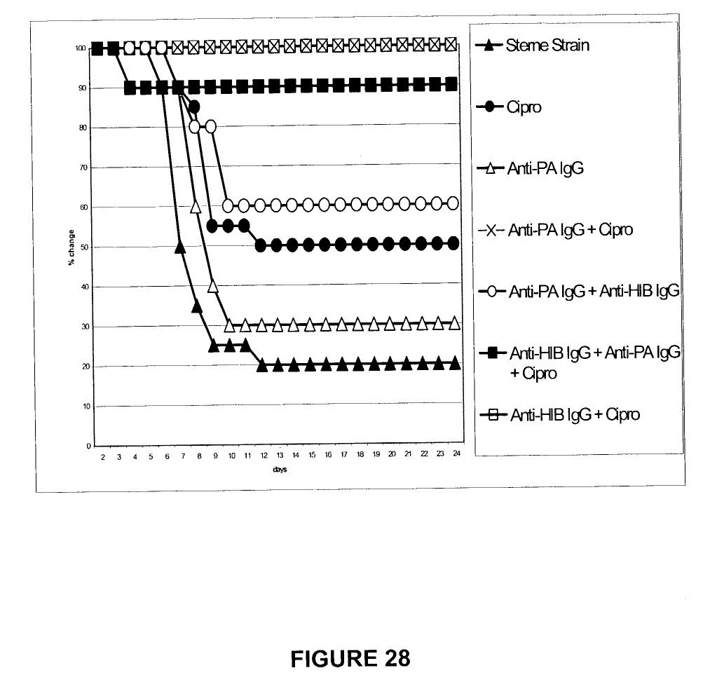

In contrast to the treatment with the antibodies or ciprofloxacin alone, the combination of the antibodies and the antibiotic unexpectedly provided full protection of mice from bacterial infection, while treatment with ciprofloxacin or anti-PA IgG alone provide only 50% or less protection (FIG. 28). Therefore, the combination of antibiotics and antibodies is a novel therapy for the treatment of anthrax infection, which provides unexpected results.

### EXAMPLE 3

The survival time of mice that were pretreated with cytokines and then infected with vaccinia virus, was determined. Vaccinia virus (strain WR), a prototypical poxvirus, was used in these assays. Infection of BALB/c mouse with orthopoxviruses has proven a good model to study pathogenesis and host immune responses to viral infections (Buller 1985). This virus has been widely used as an virological and immunological research tool and model for smallpox virus infection. (Moss, 1996; Carroll and Moss, 1997.) Balb/C mice were pretreated with the indicated cytokine two days before vaccinia virus challenge. Vaccinia virus was administered intranasally on day 0 at 9.4×10⁴ pfu, which is twice the LD50. The mice were then treated with cytokine again on days 0, 2, and 4 post-infection. Both IFN-α and IFN-γ were administered with a dose of 10⁴ U/mouse/day of cytokine. Control mice received no cytokine. The experimental and control groups each included 20 mice.

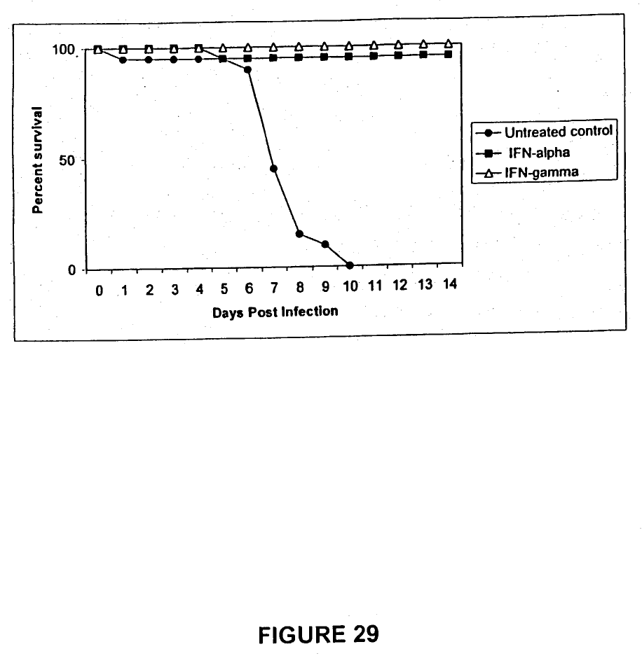

By day 10, no control mice were alive. In contrast, 95% of the IFN-α treated mice and 100% of the IFN-γ treated mice were still alive. Protection against the vaccinia virus continued until the end of the study at day 14, when 95% of the IFN-α treated mice and 100% of the IFN-γ treated mice remained alive. (FIG. 29) Therefore, IFN-α and IFN-γ protected mice:against vaccinia virus.

### EXAMPLE 4

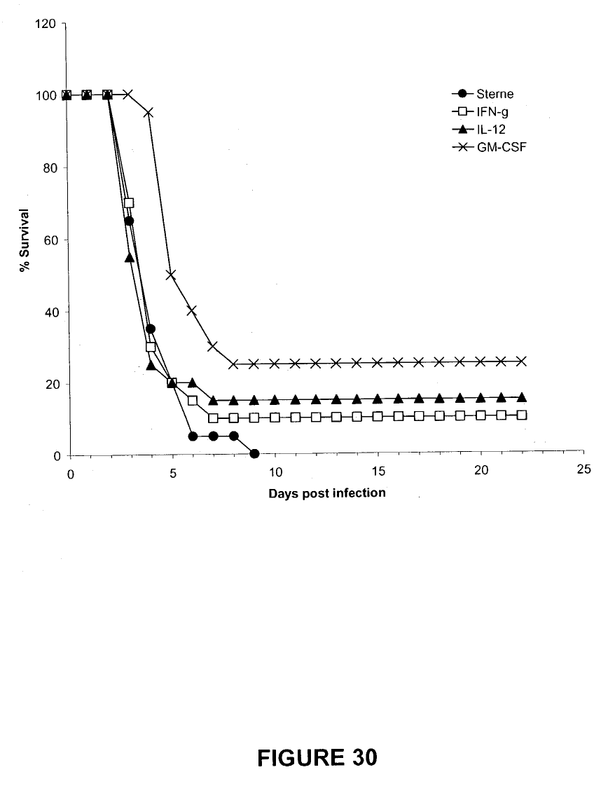

Protection of mice against B. anthracis infection by cytokine treatment was demonstrated. A/J mice were infected intraperitoneally with 5×10⁵ spores of B. anthracis (Sterne). The mice were treated with 2×10⁴ U GM-CSF, 100 ng IL-12, or 10⁴ U IFN-γ, which were administered intranasally on days −2, 0, +2, and +4 of infection. By day 9, all of the untreated mice were dead. In comparison, on day 9 10% of the mice treated with IFN-γ and 25% of the mice treated with GM-CSF were still alive. These mice remained alive until the end of the experiment on day 22. Therefore, both IFN-γ and GM-CSF provided protection against anthrax infection. (FIG. 30)

### EXAMPLE 5

The effect of bacterial cell wall from the species B. alcalophilus on survival time after anthrax infection was demonstrated.

The cell walls were prepared as follows: 150 mg of wet bacterial pellet was resuspended in 20 ml of hot 4% SDS and boiled in a covered flask, while being stirred, for 30 minutes. It was then incubated overnight at room temperature with agitation. The suspension was centrifuged at 25,000×g for 20 minutes and the supernatant was removed. The pellet was resuspended in 20 ml of hot 4% SDS and boiled in a covered flask for 15 minutes while being stirred. The procedure was repeated two times. The mixture was centrifuged at 25,000×g for 20 minutes and the supernatant was removed. The pellet was then resuspended in 30 ml of LPS-free water and centrifuged at 25,000×g for 20 minutes. Centrifugation and resuspension were repeated three times. The final pellet was resuspended in 30 ml of 2M NaCl and centrifuged at 25,000×g for 20 minutes. The pellet was resuspended in 30 ml of water and centrifuged for 20 minutes at 25,000×g. The pellet was resuspended in 5 ml of LPS-free water and the mixture was centrifuged for 20 minutes at 25,000×g. The pellet was dried using a freezedryer. The final pellet was resuspended in LPS-free water to a concentration of 1 mg dry cell wall per ml water. Cell wall was stored in 100 μL aliquots at −80 C.

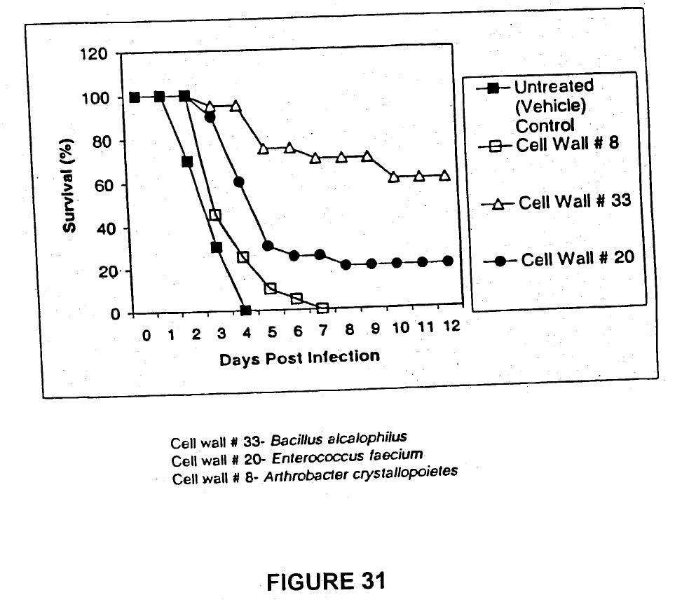

Mice were pretreated with cell walls for two days before bacterial challenge. Bacteria were administered on day 0 with 5×10⁵ spores per mice. The infected mice were then treated with cell walls on days 0, 2, and 4 post-infection with 25 μg/mouse/day. Mice were monitored for survival and bacterial counts in the spleen. No control mice, which were not treated with cell walls, were alive on day four after infection. Cell wall from Arthrobacter crystallopoietes increased survival. More than 20% of the mice treated with this cell wall were still alive on day four. Cell wall from Enterococcus faecium further increased survival of infected mice. After 12 days post-infection, 20% of the anthrax infected mice were still alive. Cell wall from Bacillus alcalophilus increased survival of anthrax-infected mice the most significantly. After 12 days, 60% of the infected mice were still alive. (FIG. 31)

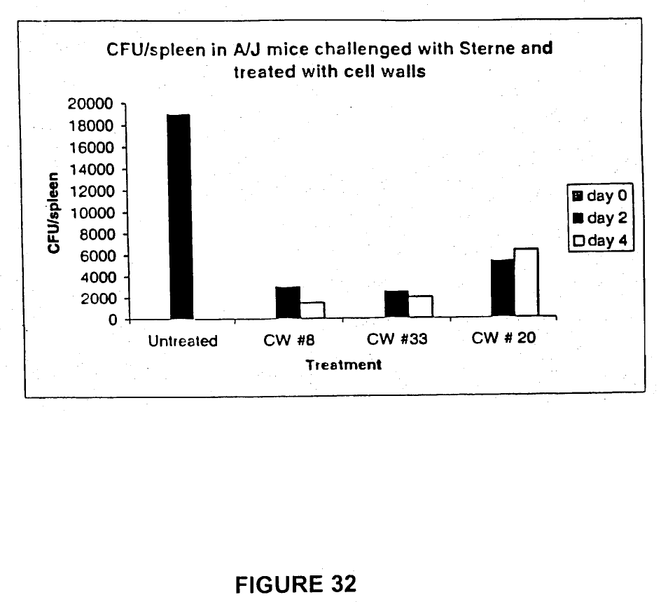

These results of survival studies correlate to the CFU determinations. Splenocytes were isolated by homogenizing spleens from three mice. The volume of the resulting splenocytes were adjusted to 10 ml. The cells were then lysed with 5% saponin (Sigma), diluted to produce several 10× dilutions in PBS, and plated on NB agar in triplicate (0.2 ml of each dilution). After overnight growth, CFU were determined. When the mice were treated with cell wall from Arthrobacter crystallopoletes, Bacillus alcalophilus, and Enterococcus faecium the CFU per spleen were dramatically decreased after two days of infection. The low levels of CFU in treated mice persisted to four days post-infection. Therefore, the cell wall of B. alcalophilus was able to prolong the survival of mice infected with anthrax. (FIG. 32)

The ability of E. faecium, S. caseolyticus, and B. stearothermoohilus to protect mice against anthrax infection can also be determined.

### EXAMPLE 6

The effect of the IFN-γ and the cell wall of B. alcalophilus, and the effect of the combination of these two components on the viral load of infected cells was determined.

Cell wall was prepared as described in Example 5. Raw 264.7 cells (2×10⁵/well) were activated by B. alcalophilus cell wall, IFN-γ, or both B. alcalophilus cell wall and IFN-γ for 20 hours. After washing twice with PBS, the activated macrophage cells were co-cultured with human 293 cells infected with vaccinia virus (MOI of 1 for 1 hour) for 20 hours. Following three cycles of freeze and thaw, the mixtures were subjected to a plaque assay. Vertical bars represent the standard error of mean from triplicate samples.

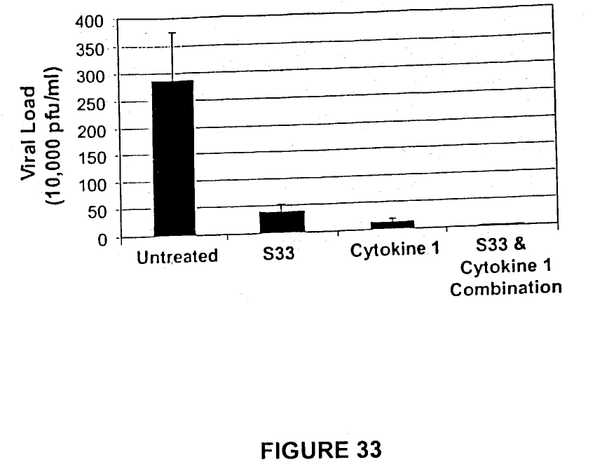

There were over 2.75×10⁶ pfu/ml produced in untreated cells. When the cells were treated with cell wall preparation (1 μg/ml) from B. alcalophilus, this number was significantly reduced to less than 0.5×10⁶ pfu/ml. Treatment with IFN-γ at 100 U/ml reduced the number of plaque forming units even further. When the cells were treated with both B. alcalophilus cell wall and IFN-γ, the levels of pfu were reduced the furthest, showing a potentially synergistic effect of the two components together. (FIG. 33)

The effect of E. faecium, S. caseolyticus, and B. stearothermoohilus on viral load in infected cells can also be determined.

### EXAMPLE 7

The cell walls of bacterial species can also be fractionated to reveal the specific components that produce the desired immune effects. The petidoglycan fraction of bacterial cell wall was isolated by the following procedure. Cell wall was prepared as described in Example 5. Thirty milligrams of lyophilized cell wall was suspended in 1 ml of 100 mM Tris-HCl (pH 7.5). α-Amylase was added to a final concentration of 100 μg/ml and incubated for two hours. The sample was then treated with a final concentration of 100 μg/ml DNase and 100 μg/ml RNase, both resuspended in 20 mM MgSO₄, for two hours at 37° C. Finally, the sample was treated with a final concentration of 100 μg/ml trypsin in 10 mM CaCl₂ for 16 hours. The enzymes were inactivated by boiling with 1% SDS for 15 minutes. The sample was then centrifuged at 40,000×g for 15 minutes. The resulting pellet was washed twice with distilled water to remove the SDS, by centrifuging at 20,000×g for 15 minutes and resuspending the pellet. The cells were then washed once in 8M LiCl and centrifuged at 20,000×g for 15 minutes. The pellet was then washed four times in distilled water. The final pellet was stored dry at either room temperature or at −20° C.

To isolate peptidoglycan, the 5 mg of the dried pellet was resuspended in 0.5-1.0 ml of hydroflouric acid (49% w/v) in 15 ml polypropylene centrifuge tubes. The suspension was incubated at 4° C. for 48 hours, followed by centrifugation and resuspension of the pellet in sterile distilled water. The suspension was then centrifuged at 30,000×g for 30 minutes. Washes in distilled water were repeated for four washes total. After the final wash, the pellet was resuspended in 100 mM Tris-HCl (pH 7.5) and washed with distilled water until the pH was neutral. A final centrifugation at 30,000×g was done for 30 minutes and the supernatant was discarded. The pellet was resuspended in 100 mM (NH₄)₂CO₃ with alkaline phosphatase (250 μg/ml) and incubated overnight (16 hours) at 37° C. After incubation, the alkaline phosphatase enzyme was inactivated by boiling for five minutes. The resulting pure peptidoglycan was washed two times in sterile distilled water and stored at 20° C.

Lipoteichoic acid purification was achieved by suspending a defrosted aliquot of bacterial cell wall (see Example 5) in 0.1 M sodium acetate that has been adjusted to pH 4.5 with acetic acid, to a final concentration of 800 mg/ml, and mixed with equal volume of n-butanol for 30 minutes at room temperature, with stirring. This mixture was then centrifuged at 13,000×g for 20 minutes. The aqueous phase was lyophilized The pellet was then resuspended with chromatography start buffer (15% n-propanol in 0.1 M ammonium acetate, pH 4.7) and centrifuged at 45,000×g for 15 minutes. The supernatant was fractionated by hydrophobic interaction chromatography (HIC) on an octyl-Sepharose column (FPLC). The fractions were eluted with increasing concentrations of propanol (0-60%) in 0.1 M ammonium acetate buffer. The resulting LTA was quantitated by measuring the phosphate.

An important practical consideration in preparing and testing PGN, LTA, or muramyl peptides is to avoid contamination by LPS from Gram-negative bacteria, which share most of the biological activities of muramyl peptides. All the above preparations were confirmed for the absence of Gram-negative endotoxin using E-TOXATE® (Sigma) and stored at −20° C.

### EXAMPLE 8

Cell Wall/PGN-Induced Antiviral Activity

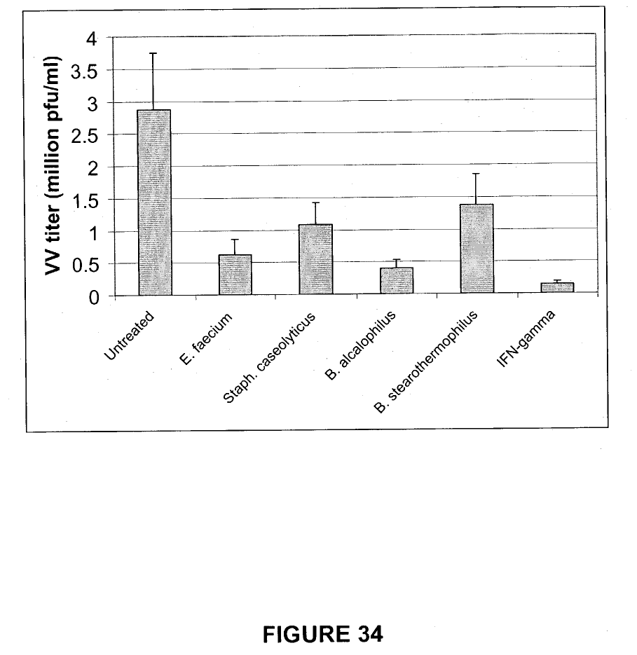

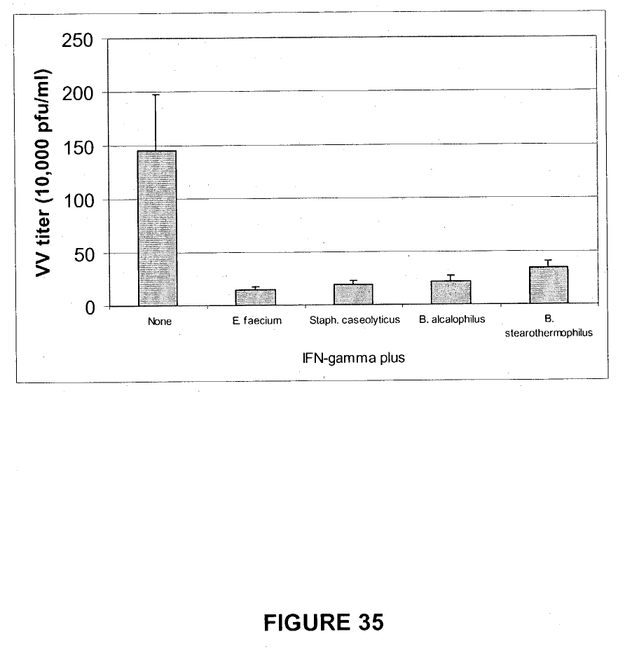

RAW 264.7 cells respond well to IFN-γ and/or LPS stimulation for NO production. It has been demonstrated that IFN-γ is capable of enhancing the antiviral activity of macrophage cells. Treatment of RAW 264.7 cells with IFN-γ, leads to a 95% reduction in VV titer. (FIG. 34) This corresponds to a significant increase in NO release by macrophages. VV titers in samples treated with whole cell walls from E. faecium, Staph caseolyticus, B. alcalophilus, and B. stearothermophilus were reduced to 22, 38, 14, and 44% of that in control, respectively. (FIG. 34) VV titers in samples treated with each of these cell walls plus IFN-γ were reduced to 10, 13, 15, and 34% of that in sample treated with IFN-γ alone, respectively. (FIG. 35) Peptidoglyan (PGN) of B. alcalophilus was further assayed for its effect on the inhibition of VV replication in the same assay. As shown in FIG. 8, PGN, within the test range (1-30 μg/ml), reduced VV titer to 10-20% of that in control. Based on these results, it is concluded that a combination of soluble cell wall preparations and IFN-γ significantly enhances the antiviral activity of murine macrophages. In addition, PGN of B. alcalophilus functions as a better or equally good antiviral inducer of murine macrophages compared to sample treated with CW. This is supported by NO stimulation data (FIG. 9), showing that PGN in the test range induces as much as or even slightly higher levels of NO production compared to sample treated with CW.

In addition, the cell walls from E. faecium, Staph caseolyticus, and B. alcalophilus, three of the above mentioned candidates, are able to significantly enhance the cytolytic activities of NK/NKT cells from human peripheral blood mononuclear cells (PBMC) against K-562 tumor target cells. Second, intranasally administered IFN-γ has been shown to be able to confer complete protection on BALB/c mice against lethal VV (strain WR) challenge. Third, intraperitoneally administered cell wall of E. faecium and B. alcalophilus are able to protect BALB/c mice from lethal Bacillus anthrncis (strain Stern) challenge, with survival rate of 20% to 60%, compared to that of untreated control (0%).

In addition, protection against viral infection by E. faecium, S. caseolyticus, and B. stearothermoohilus, as well their peptidoglycan, lipoteichoic acid and muramyl peptide fractions, can also be determined.

### EXAMPLE 9

In Vivo Activation of Mice with Bacillus alcalophilus Peptidoglycan, and Synthetic Muramyl Peptide [MDP-Lys (L18)]

The optimal dosage of compound administration for the best activation of macrophages and NK cells can be determined. Groups of 25 BALB/c mice can be treated once intranasally with different amounts of PGN (1, 10, 100, or 1000 μg), and MDP-Lys(L18) (1, 10, 100 μg) in a 20-μl volume. Control groups (5 mice each) can be treated daily for four times with PBS, IFN-γ (intranasally,10⁴ IU), or CW (i.p., 50 μg) daily for 4 days. These doses of MDP-Lys(L18) and PGN based on the previously published reports (Azuma et al., 1988; Ikeda et al., 1985; Guencheva et al., 1992; Mashihi et al., 1984, 1989; Kende et al., 1988; Davidkova et al., 1992; Bogdanov et al., 1991) can be used. Five mice from each group can be sacrificed daily for five days by cervical dislocation following i.p. injection of ketamine (100 mg/kg). Cytokine- and CW-treated groups are sampled on day 5. Spleen and peritoneal cavity fluid is aseptically collected for isolation of splenocyte and peritoneal macrophages following commonly used procedures (Kruisbeek, 1999; Fortier, 1994).

Splenocyte from treated and untreated mice are be assayed for NK cell cytotoxicity in a 4-h chromium-51 (⁵¹Cr) release assay using YAC-1 tumor cells as a target (Brunner et al., 1968). Furthermore, peritoneal macrophages from treated and untreated mice are assayed for NO release following 48-h incubation at 37° C., and extrinsic antiviral against VV-infected cells (human 293 or murine NIH/3T3 cells) according to published procedures (Ding et al., 1988; Ikeda et al., 1985). One the optimum dose of each compound is selected, similar experiments are conducted to test if IFN-γ synergies PGN or MDP-Lys(L18) for activation of macrophages and NK cells (>10 group×25). Statistical analysis (ANOVA and student's t-test) is used throughout all of the studies to determine significant differences between the experimental and control groups.

### EXAMPLE 10

Cytokine expression profiles of mouse organs following cell wall or cell wall component treatment and/or lethal viral challenge can be performed using ribonuclease protection assay (RPA) for tissues/organs or ELISA for plasma, from portions of samples from days 2 and 5 post infection. Cytokines that can be assayed include IL-2, IL-12, IL-15, II-18, IFN-γ, IFN-α/β, TNF-α, IL-1, and IL-6.

The chemokine expression profile of mouse organs following treatment with CW (or CW components) and/or lethal viral challenge can be determined. The selected chemokines include RANTES, IL-8, MIP-1 (microphage inflammatory protein 1), MCP-1, MCP-3 (monocyte chemoattractant protein), Crg-2, and I-309.

### EXAMPLE 11

The minimum dose of IFN-γ and cell wall or cell wall fraction can be determined. In addition, minimum frequency required to achieve the best protection on mice challenged with lethal dose of VV can also be determined.

Groups of 10 mice can be infected intranasally with VV and then treated with PGN33/IFN-γ (1 dose/day) on days −1, +1, +3, +5; 0, +1, +3, +5; +1, +3, +5; +1, +3; or +1, +2. Mice are monitored for illness, weight loss, and mortality for 3 weeks.

Cidofovir has been shown to be able to protect BALB/c mice from respiratory infections of VV and CPV. The selected combination of IFN-γ and cell wall or cell wall fraction can protect VV- and CPV-infected mice differently. Therefore, it is necessary to compare the protective effect of the selected combination in two mouse models of respiratory orthopoxvirus infection. Groups of 20 mice can be infected intranasally with 10⁶ pfu CPV or VV per mouse, then treated with either cidofovir (day 1, 40 mg/kg/day, intranasally for CPV, or 30 mg/kg on days 1 and 4, subcutaneously for VV), the peptidoglycan fraction of B. alcalophilus plus B. alcalophilus/IFN-γ, or PBS following the previously determined regimen of administration. Animals are monitored daily for weight loss, illness, and survival rate for 21 days. Five mice from each group are sacrificed on days 2 and 5; and organ virus titers and cytokine/chemokine profiles are determined as in Example 8.

### EXAMPLE 12

The effects of ciprofloxacin in combination with sheep antibodies against protective antigen and live bacteria as a treatment for B. anthracis infection were demonstrated. Twelve-week-old female DBA/2 mice were inoculated with 1×10⁷ spores of the Bacillus anthracis Sterne strain by intraperitoneal route. Five hours after infection, the mice were injected i.p. with 10 mg/kg of anti-PA IgG, anti-BA IgG. On days 2 and 3 the mice were given two injections per day (morning and late afternoon) of the antibodies. On days 4 to 10, mice were injected with antibodies once a day. Ciprofloxacin was administered (50 mg/kg) subcutaneously once a day on days 2 to 10. The delayed treatment (D) groups were treated with anti-PA antibodies once a day on days 2 to 10. The mice were monitored daily.

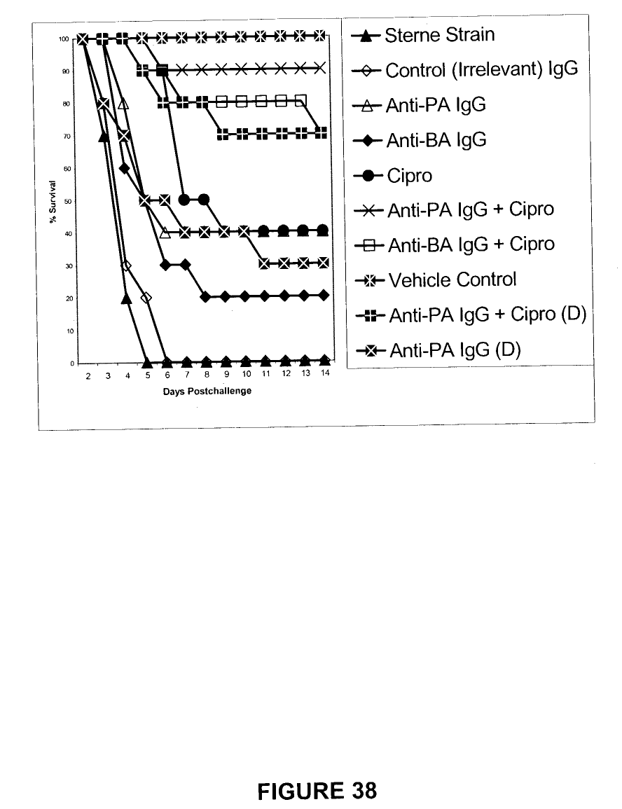

As shown in FIG. 38, all of the mice died by 6 days after infection with B. anthracis when the mice were untreated or treated with only an irrelevant IgG. Treatment with antibodies raised against the whole bacteria alone resulted in the survival of 20% of the mice by 14 days after infection. Similar results were seen when the infected mice were treated with anti-PA antibody after a delay in treatment (30% survival) or with ciprofloxacin alone or anti-PA IgG alone (40% survival). When the infected mice were treated with anti-PA IgG and ciprofloxacin in a delayed treatment, 70% survived after 14 days. A similar number of the infected mice survived when they were treated with anti-whole bacteria IgG and ciprofloxacin, and the treatment was not delayed. Surprisingly, when the mice were treated with sheep anti-PA IgG plus ciprofloxacin without any delay, 90% of the mice survived. This number is greater than the number expected to survive when treated with anti-PA IgG alone (40%) or ciprofloxacin alone (40%), and demonstrates a synergistic effect of the sheep anti-PA IgG and ciprofloxacin.

References

The following references are cited herein. The entire disclosure of each reference is relied upon and incorporated by reference herein.

1. Aderem, A. and Ulevitch, R. J. 2001 Toll-like receptors in the induction of the innate immune response. Nature, vol. 406, pp. 782-787.

2. Akarid, K., Sinet, M., Desforges, B., and Gougerot-Pocidalo, M. A. 1995. Inhibitory effect of nitric oxide on the replication of a murine retrovirus in vitro and in vivo. J Virol. 69(11):7001-5.

3. Angel J B, High K, Rhame F, Brand D, Whitmore J B, Agosti J M, Gilbert M J, Deresinski S. 2000 Phase III study of granulocyte-macrophage colony-stimulating factor in advanced HIV disease: effect on infections, CD4 cell counts and HIV suppression. Leukine/HIV Study Group. AIDS 14:4 387-95.

4. Armitage J O. 1998 Emerging Applications of Recombinant Granulocyte-Macrophage Colony-Stimulating Factor. J Am Soc Hem 92:12 4491-4508.

5. Azuma, I., Iida, J., Nishimura, K., Ishihara, C., Tokura, S., and Yamamura, Y. 1988 Prevention of Sendai virus infection with synthetic MDP and chitin derivatives in mice. Advances in Biosciences 68, 29-37.

6. Bektemirov, T. A, Burgasova, M. P., Kuznetsov, V. P, Rozina, E. E., and Andzhaparidze, O. G. 1980. Smallpox vaccination with protection by interferon and an interferon inducer. Vopr. Virusol: Smallpox vaccination with protection by interferon and an interferon inducer. Vopr Virusol. January-February;(1):76-8.

7. Biron, C. A. 1997. Activation and function of natural killer cell responses during viral infections. Curr Opin Immunol 1997. 9(1):24-34.

8. Biron, C. A. 1998. Role of early cytokines, including alpha and beta interferons (IFN-alpha/beta), in innate and adaptive immune responses to viral infections. Semin Immunol 10(5):383-90.

9. Biron, C. A., Nguyen, K. B., Pien, G. C., Cousens, L. P., and Salazar-Mather, T. P. 1999. Natural killer cells in antiviral defense: function and regulation by innate cytokines. Annu Rev Immunol 17:189-220.

10. Brutkiewicz, R. R., Klaus, S., and Welsh, R. M. 1992. Window of vulnerability of vaccinia virus-infected cells to natural killer (NK) cell-mediated cytolysis correlates with enhanced NK cell triggering and is concomitant with a decrease in H-2 class I antigen expression. Nat Immun 11 :203-214.

11. Bermudez L E, Martinelli J, Petrofsky M, Kolonoski P, Young L S. 1994 Recombinant granulocyte-macrophage colony-stimulating factor enhances the effects of antibiotics against Mycobacterium avium complex infection in the beige mouse model. J Infect Dis 169(3):575-80.

12. Brunner, K. T., Mauel, J., Cerottini, J. C., and Chapuis, B. 1968 Quantitative assay of the lytic action of immune lymphoid cells on 51-Cr-labelled allogeneic target cells in vitro; inhibition by isoantibody and by drugs. Immunology 14, 181-196.

13. Buller, R. M. 1985. The BALB/c mouse as a model to study orthopoxviruses. Curr Top.Microbiol Immunol 122, 148-153.

14. Carroll, M. W., and Moss, B. 1997. Poxviruses as expression vectors. Curr Opin Biotechnol. 8(5):573-7.

15. Claus, D. and Berkely, R. C. W. in Bergey's Manual of Systematic Bacteriology, vol. 2, 1986, John P. Butler, Ed., (Williams & Wilkins 1986), pp. 1105-1139.

16. Croen, K. D. 1993. Evidence for antiviral effect of nitric oxide. Inhibition of herpes simplex virus type 1 replication. J Clin Invest. 91(6):2446-52.

17. Davidkova, G., Popova, P., Guencheva, G., Bogdanov, A., Pacelli, E., Auteri, A., and Mincheva, V. 1992 Endogenous production of tumor necrosis factor in normal mice orally treated with Deodan—a preparation from Lactobacillus bulgaricus“LB51” Int.J Immunopharmacol. 14, 1355-1362.

18. Dierdorf R, Kreuter U, Jones T C. 1997 A Role for Granulocyte-Macrophage Colony-Stimulating Factor (GM-CSF) in the Treatment of Neutropenic Patients with Pneumonia. Braz J Infect Dis 1:2 68-76.

19. Ding, A. H., Nathan, C. F., and Stuehr, D. J. 1998. Release of reactive nitrogen intermediates and reactive oxygen intermediates from mouse peritoneal macrophages. Comparison of activating cytokines and evidence for independent production. J Immunol. 141(7):2407-12.

20. Fortier, A. H. 1994 Isolation of murine peritoneal macrophages. In “Current Protocols in Immunology” (J. E. Coligan, A. M. Kruisbeek, D. H. Margulies, E. M. Shevach, and W. Strober, Eds.), Vol. 3, p.14.1.1-14.1.3. John Wiley & Sons, Inc., USA.

21. Guencheva, G., Popova, P., Davidkova, G., Mincheva, V., Mihailova, S., Bogdanov, A., Pacelli, E., and Auteri, A. 1992 Determination of cytokine release after in vivo and in vitro administration of Deodan (a preparation from Lactobacillus bulgaricus“LB51”) by the rabbit pyrogen test. Int.J Immunopharmacol. 14, 1429-1436.

22. Guidotti, L. G., and Chisari, F. V. 2001. Noncytolytic control of viral infections by the innate and adaptive immune response. Annu Rev Immunol. 19:65-91.

23. Harris, N., Buller, R. M., and Karupiah, G. 1995. Gamma interferon-induced, nitric oxide-mediated inhibition of vaccinia virus replication. J Virol. 69(2):910-15.

24. Herbein, G. 1997. Cytokines, viruses and macrophages: an interactive network. An immune dysregulation involving the members of the tumor necrosis factor (TNF) receptor superfamily could be critical in AIDS pathogenesis. Pathol Biol (Paris). 45(2):115-25.

25. Hsu N, Young L S, Bermudez L E. 1995 Response to stimulation with recombinant cytokines and synthesis of cytokines by murine intestinal macrophages infected with the Mycobacterium avium complex. Infect Immun 63(2) 528-33.

26. Ikeda, S., Negishi, T., and Nishimura, C. 1985 Enhancement of non-specific resistance to viral infection by muramyldipeptide and its analogs. Antiviral Res 5, 207-215.

27. Inglesby T V, Henderson D A, Bartlett J G, Ascher M S, Eitzen E, Friedlander A M, Hauer J, McDade J, Osterholm M T, O'Toole T, Parker G, Perl T M, Russell P K, and Tonat K Anthrax as a biological weapon: medical and public health management, Working Group on Civilian Biodefense, JAMA 1999 281 1735-1745 (1999).

28. Jernigan J A, et al., Bioterrorism-related inhalational anthrax: the first 10 cases reported in the United States, Emerg Infect Dis 2001 7(6) 933-44 (2001).

29. Karpov, A. V. 2001. Endogenous and exogenous interferons in HIV-infection. Eur J Med Res. 6(12):507-24.

30. Karupiah, G., Woodhams, C. E., Blanden, R. V., and Ramshaw, I. A. 1991. Immunobiology of infection with recombinant vaccinia virus encoding murine IL-2. Mechanisms of rapid viral clearance in immunocompetent mice. J lmmunol.147(12):4327-32.

31. Karupiah, G., and Harris, N. 1995. Inhibition of viral replication by nitric oxide and its reversal by ferrous sulfate and tricarboxylic acid cycle metabolites. J Exp Med. 181(6):2171-9.

32. Kende, M., Lupton, H. W., and Canonico, P. G. 1988 Treatment of experimental viral infections with immunomodulators. Advances in Biosciences 68, 51-63.

33. Kholer, G. and Milstein, C., Continuous cultures of fused cells secreting antibody of predefined specificity. Nature, 256: 495 (1975).

34. Kontsek, P., and Kontsekova, E. 1997. Forty years of interferon. Acta Virol 41(6):349-53.

35. Kruisbeek, A. 1998 Isolation of mouse mononuclear cells. In “Current Protocols in Immunology” (J. E. Coligan, A. M. Kruisbeek, D. H. Margulies, E. M. Shevach, and W. Strober, Eds), Vol. 1, p. 3.1.2-3.1.5. John Wiley & Sons, Inc., USA.

36. Krusteva, e. Hristova, S. Damyanov, D., Bogdanov, A. Altaparmakov, I, and Pcelli, E. 1998 Clinical Study of the Effect of the Preparation Deodan on Leukopenia, Induced by Cytostatics. Int. J. Immunopharmac. 19: 487-492.

37. Kwon, N. S., Stuehr, D. J., and Nathan, C. F. 1991. Inhibition of tumor cell ribonucleotide reductase by macrophage-derived nitric oxide. J Exp Med. 174(4):761-7.

38. Lai, C. L., and Wu, P. C. 1997. Antiviral treatment for chronic hepatitis B. Hong Kong Med J 3(3):289-296.

39. Lepoivre, M., Fieschi, F., Coves, J., Thelander, L., and Fontecave, M. 1991. Inactivation of ribonucleotide reductase by nitric oxide. Biochem Biophys Res Commun. 179(1):442-8.

40. Lidbury, B. A., Ramshaw, I. A., and Sambhi“S. K. 1995. The role for host-immune factors in the in vivo antiviral effects of tumour necrosis factor. Cytokine.7(2):157-64.

41. Liles W C. 2001 Immunomodulatory approaches to augment phagocyte-mediated host defense for treatment of infectious diseases. Semin Respir Infect 16(1):11-7.

42. Lin, Y. L., Huang, Y. L., Ma, S. H., Yeh, C. T., Chiou, S. Y,, Chen, L. K., and Liao, C. L. 1997. Inhibition of Japanese encephalitis virus infection by nitric oxide: antiviral effect of nitric oxide on RNA virus replication. J Virol. 71(7):5227-35.

43. Luh, K -T, Yang, P -C, Kuo, S -H., chang, D -B., Yu,C -J., and Lee, L -N. 1992 Comparison of OK-432 and Mitomycin C Pleurodesis for Malignant Pleural Effusion Caused by Lung Cancer. Cancer 69:674-679.

44. MacMicking, J., Xie Q. W., and Nathan, C. 1997. Nitric oxide and macrophage function. Annu Rev Immunol. 15:323-50.

45. Martinez, B. M., Smee, D. F., Kefauver, D., Thompson, E., and Huggins, J. W. 2000. Cidofovir protects mice against lethal aerosol or intranasal cowpox virus challenge. J Infect Dis 181(1):10-9.

46. Masihi, K. N., Brehmer, W., Lange, W., Ribi, E., and Schwartzman, S. 1984 Protective effect of muramyl dipeptide analogs in combination with trehalose dimycolate against aerogenic influenza virus and Mycobacterium tuberculosis infections in mice. J Biol Response Mod. 3, 663-671.

47. Melkova, Z., and Esteban, M. 1994. Interferon-gamma severely inhibits DNA synthesis of vaccinia virus in a macrophage cell line. Virology. 198(2):731-5.

48. Melian, E. B., and Plosker, G. L. 2001. Interferon alfacon-1: a review of its pharmacology and therapeutic efficacy in the treatment of chronic hepatitis C. Drugs. 61(11):1661-91.

49. Moradpour, D., and Blum, H. E. 1999. Current and evolving therapies for hepatitis C. Eur J Gastroenterol Hepatol 11(11):1199-202.

50. Morahan, P. S., Connor, J. R., and Leary, K. R. 1985. Viruses and the versatile macrophage. Br Med Bull. 41(1):15-21.

51. Moss, B. 1996. Genetically engineered poxviruses for recombinant gene expression, vaccination, and safety. Proc Natl Acad Sci U S A. 93(21):11341-48.

52. Okawa, T., Niibe, H., Arai, T., Sekiba, K., Noda, K., Takeuchi, S., Hashimoto, S., and Ogawa N. 1993 Effect of LC9018 Combined with Radiation Therapy on Carcinoma of the Uterine Cervix. Cancer 72: 1949-1954.

53. Pugin, J., Heumann, I. D., Tomasz, A., Kravchenko, V. V., Akamatsu, Y., Nishijima, M., Glauser, M. P., Tobias, P. S., and Ulevitch, R. J. (1994). CD14 is a pattern recognition receptor. Immunity. 1, 509-516.

54. Ramshaw, I. A., Ramsay, A. J., Karupiah, G., Rolph, M. S., Mahalingam, S., and Ruby, J. C. 1997. Cytokines and immunity to viral infections. Immunol Rev 159:119-35.

55. Reiss, C. S., and Komatsu, T. 1998. Does nitric oxide play a critical role in viral infections? J Virol. 72(6):4547-51.

56. Rolph, M. S., Cowden, W. B., Medveczky, C. J., and Ramshaw, I. A. 1996. A recombinant vaccinia virus encoding inducible nitric oxide synthase is attenuated in vivo. J Virol. 70(11):7678-85.

57. Rolph, M. S., Ramshaw, I. A., Rockett, K. A., Ruby, J., and Cowden, W. B. (1996). Nitric oxide production is increased during murine vaccinia virus infection, but may not be essential for virus clearance. Virology 217, 470-477.

58. Sambhi, S. K, Kohonen-Corish, M. R., and Ramshaw, I. A. 1991. Local production of tumor necrosis factor encoded by recombinant vaccinia virus is effective in controlling viral replication in vivo. Proc Natl Acad Sci U S A. 88(9):4025-9.

59. Singh P P, Singh S. 2001 Protection of mice from malaria after co-administration of recombinant mouse granulocyte-macrophage colony-stimulating factor and methionine-enkephalin. Eur Cytokine Netw 12(3):528-36.

60. Takeuchi, O. and Akira, S. 2001 Toll-like receptors; their physiological role and signal transduction system. Int. Immunopharm., vol. 1, pp. 625-635.

61. Van den Broek, M. F., Muller, U., Huang, S., Zinkernagel, R. M., and Aguet, M. 1995. Immune defence in mice lacking type I and/or type II interferon receptors. Immunol.Rev. 148:5-18.

62. Vazquez J A, Hidalgo J A, De Bono S. 2000 Use of sargramostim (rh-GM-CSF) as adjunctive treatment of fluconazole-refractory oropharyngeal candidiasis in patients with AIDS: a pilot study. HIV Clin Trials 1(3):23-9.

63. Welsh, R. M. 1986. Regulation of virus infections by natural killer cells. A review. Nat Immun Cell Growth Regul. 5(4):169-99.

64. Xing, Z, and Wang, J. 2000. Consideration of cytokines as therapeutics agents or targets. Curr Pharm Des. 6(6):599-611.

65. Xing, Z. 2000. Current understanding of macrophage type 1 cytokine responses during intracellular infections. Histol Histopathol. 15(1):199-205.

66. Yamamoto Y, Klein T W, Tomioka M, Friedman H 1997 Differential effects of granulocyte/macrophage colony-stimulating factor (GM-CSF) in enhancing macrophage resistance to Legionella pneumophila vs Candida albicans. Cell Immunol 1997 176:1 75-81.

67. Zurzrock, R., Rosenblum M. G., Sherwin, S. A., Rios, A., Talpz, M. Quesada, J. R., and Gutterman, J. U. 1985 Pharmacokinetics, single-dose tolerance, and biological activity of recombinant gamma-interferon in cancer patients. Cancer Res. 45: 2866-72.

What is claimed is:

1. A treatment for the effects of biological weapons comprising a rapid-acting, broad spectrum therapy.

2. A treatment for anthrax infection comprising administering a composition comprising antibiotic and antibodies against antigens of B. anthracis to a person exposed to a biological threat agent.

3. The treatment as claimed in claim 2, wherein the antibiotic and the antibodies are administered together.

4. The treatment as claimed in claim 2, wherein the antibiotic is administered before the antibody.

5. The treatment as claimed in claim 2, wherein the antibodies are administered before the antibiotic.

6. The treatment as claimed in claim 2, wherein the antibodies are raised against protective antigen (PA) of anthrax or against live or killed bacteria.

7. The treatment as claimed in claim 2, wherein the antibiotics are fluoroqinalones, tetracyclines, or β lactams.

8. The treatment as claimed in claim 2, wherein the antibiotic is ciprofloxacin.

9. A treatment for an animal infected with a poxvirus or with anthrax comprising administering a composition comprising an immunostimulator to the infected animal and the increasing the survival time of the animal.

10. The treatment as claimed in claim 9, wherein the treatment is a therapeutic method and is administered after the animal is infected.

11. The treatment as claimed in claim 9, wherein the treatment is a prophylactic treatment administered before the animal is infected.

12. The treatment as claimed in claim 9, wherein the animal is at risk for harmful effects of a vaccine to smallpox.

13. The treatment as claimed in claim 9 wherein the composition comprises at least one of a cytokine, a bacterial cell wall, or a fraction of a bacterial cell wall.

14. The treatment as claimed in claim 13, wherein the immunostimulator is administered by inhaling the immunostimulator.

15. The treatment as claimed in claim 13, wherein the cytokine is at least one of IFN-α, IFN-γ, and GM-CSF.

16. The treatment as claimed in claim 13, wherein the bacterial cell wall is the cell wall from at least one of B. alcalophilus, E. faecium, S. caseolyticus, or B. stearothermoohilus.

17. The treatment as claimed in claim 13, wherein the fraction of the bacterial cell wall is the peptidoglycan, lipoteichoic acid, or muramyl peptide fraction of the cell wall of B. alcalophilus, E. faecium, S. caseolyticus, or B. stearothermophilus.

18. The treatment as claimed in claim 13, wherein the immunostimulator is the combination of IFN-γ and the cell wall of B. alcalophilus, E. faecium, S. caseolyticus, or B. stearothermophilus.

19. The treatment as claimed in claim 13, wherein the poxvirus is vaccinia virus.

20. The treatment as claimed in claim 13, wherein the poxvirus is smallpox virus.

21. The treatment as claimed in claim 9, wherein the animal is a human.

22. The treatment as claimed in claim 13, wherein the cytokine is IFN-α or IFN-γ and the infection is an infection of smallpox or human monkeypox.

23. A treatment as claimed in claim 13, wherein the cytokine is IFN-γ, the bacterial cell wall is the cell wall of B. alcalophilus, and the infection is an infection of smallpox.

24. The treatment as claimed in claim 23, wherein the cell wall is the peptidoglycan fraction of B. alcalophilus.

25. The treatment as claimed in claim 13, wherein the cytokine is GM-CSF, the cell wall is the cell wall of B. alcalophilus, and the infection is anthrax infection.
